(19) United States
(12) Patent Application Publication
(10) Pub. No.: US 2023/0227777 A1
(43) Pub. Date: Jul. 20, 2023

(54) METHODS FOR ISOLATING GAMMA DELTA T CELLS

(71) Applicant: GAMMADELTA THERAPEUTICS LIMITED, White City Place, London (GB)

(72) Inventors: Madeline DALZIEL, White City Place, London (GB); Andrew HUTTON, White City Place, London (GB); Sean O'FARRELL, White City Place, London (GB); Oliver NUSSBAUMER, White City Place, London (GB)

(73) Assignee: GAMMADELTA THERAPEUTICS LIMITED, White City Place, London (GB)

(21) Appl. No.: 17/998,766

(22) PCT Filed: May 12, 2021
(86) PCT No.: PCT/GB2021/051144

§ 371 Date: Nov. 14, 2022

(30) Foreign Application Priority Data
May 12, 2020 (GB) 2006989.4

(51) Int. Cl. C12N 5/0783; A01N 1/02

(52) U.S. Cl. CPC C12N 5/0636; A01N 1/0284; A01N 1/0221; C12N 2501/2301; C12N 2501/2302; C12N 2501/2304; C12N 2501/2315; C12N 2501/2321; C12N 2501/24; C12N 2500/84

(57) ABSTRACT

The invention relates to methods for the isolation of non-haematopoietic tissue-resident lymphocytes, particularly γδ T cells. Such γδ T cells include non-Vδ2 cells, e.g. Vδ1, Vδ3 and Vδ5 cells and such non-haematopoietic tissues include skin and gut. It will be appreciated that such isolated non-haematopoietic tissue-resident lymphocytes find great utility in adoptive T cell therapies, chimeric receptor therapies and the like. Also provided are methods for expanding isolated tissue-resident lymphocytes, particularly methods for isolating and expanding γδ T cells. The present invention also relates to both individual cells and populations of cells produced by the methods described herein.

### FIELD OF THE INVENTION

The invention relates to methods for the isolation of non-haematopoietic tissue-resident lymphocytes, particularly γδ T cells. Such γδ T cells include non-Vδ2 cells, e.g. Vδ1, Vδ3 and Vδ5 cells and such non-haematopoietic tissues include skin and gut. It will be appreciated that such isolated non-haematopoietic tissue-resident lymphocytes find great utility in adoptive T cell therapies, chimeric receptor therapies and the like. The present invention also relates to both individual cells and populations of cells produced by the methods described herein.

### BACKGROUND OF THE INVENTION

The growing interest in T cell immunotherapy for cancer has focused on the evident capacity of subsets of CD8+ and CD4+ αβ T cells to recognize cancer cells and to mediate host-protective functional potentials, particularly when de-repressed by clinically mediated antagonism of inhibitory pathways exerted by PD-1, CTLA-4, and other receptors. However, αβ T cells are MHC-restricted, which can lead to graft versus host disease.

Gamma delta T cells (γδ T cells) represent a subset of T cells that express on their surface a distinct, defining γδ T-cell receptor (TCR). This TCR is made up of one gamma (γ) and one delta (δ) chain. Human γδ TCR chains are selected from three main δ chains, Vδ1, Vδ2 and Vδ3 and six γ chains. Human γδ T cells can be broadly classified based on their TCR chains, as certain γ and δ types are found on cells more prevalently, though not exclusively, in one or more tissue types. For example, most blood-resident γδ T cells express a Vδ2 TCR, for example Vγ9Vδ2, whereas this is less common among tissue-resident γδ T cells, which more frequently use Vδ1 in skin and Vγ4 in the gut.

The majority of methods for isolating lymphocytes has depended on isolating those cell types from the blood. Non-haematopoietic tissue resident lymphocytes, such as γδ T cells, may have properties especially suitable for certain applications, such as for targeting non-haematopoietic tumors and other targets. However, isolating such tissue resident lymphocytes in clinically relevant quantities has remained a challenge, especially as clinical doses ranging from 10⁸ cells upwards are required for many indications. Importantly, significant cell loss during production means even more starting cells must be generated.

Because non-haematopoietic tissue-resident lymphocytes, particularly γδ T cells, are not easily obtainable in high numbers, they have not been well characterized or studied for therapeutic applications. Therefore, there is a need in the field for methods to isolate non-haematopoietic tissue-resident lymphocytes, in particular γδ T cells, to quantities sufficient for further expansion and potentially adapt as therapies, e.g., as adoptive T cell therapies.

Clark et al. (2006) J. Invest. Dermatol. 126(5): 1059-70 describes a method of isolating skin resident T cells from normal and diseased skin. However, the methods described therein are unsuitable for clinical use due the presence of animal products but especially due to the relatively low yield of cells isolated, namely less than 10⁶ cells per cm² tissue. The method described in Clark et al. uses minced samples which results in deliberate disruption to the structural integrity of the tissue sample. WO2017072367 and WO2018/202808 relate to methods of expanding non-haematopoietic tissue-resident γδ T cells in vitro by culturing lymphocytes obtained from non-haematopoietic tissue in the presence of at least Interleukin-2 (IL-2) and/or Interleukin-15 (IL-15). WO2015189356 describes a composition for expanding lymphocytes obtained from a sample obtained by aphaeresis comprising at least two types of cytokines selected from IL-2, IL-15 and IL-21. Therefore, there still remains a need for a method of isolating tissue-resident non-haematopoietic lymphocytes, such as from skin, that yields a greater amount of cells that are suitable for clinical use.

### SUMMARY OF THE INVENTION

According to a first aspect of the invention, there is provided a method for the isolation of lymphocytes from a non-haematopoietic tissue sample comprising the steps of:
(i) culturing the non-haematopoietic tissue sample in the presence of Interleukin-1β (IL-1β); and
(ii) collecting a population of lymphocytes cultured from the non-haematopoietic tissue sample.

According to a further aspect, there is provided a method for the isolation of γδ T cells from a non-haematopoietic tissue sample comprising the steps of:
(i) culturing the non-haematopoietic tissue sample in the presence of IL-1β; and
(ii) collecting a population of γδ T cells cultured from the non-haematopoietic tissue sample.

According to a further aspect, there is provided a method for the isolation and expansion of lymphocytes from a non-haematopoietic tissue sample comprising the steps of:
(i) isolating a population of lymphocytes from the non-haematopoietic tissue sample according to the method described herein; and
(ii) further culturing said population of lymphocytes for at least 5 days to produce an expanded population of lymphocytes.

According to a further aspect, there is provided a method for the isolation and expansion of γδ T cells from a non-haematopoietic tissue sample comprising the steps of:
(i) isolating a population of γδ T cells from the non-haematopoietic tissue sample according to the method described herein; and
(ii) further culturing said population of γδ T cells for at least 5 days to produce an expanded population of γδ T cells.

### BRIEF DESCRIPTION OF THE FIGURES

FIG. 1: Percentage (%) γδ T cells of live cells (A) and total γδ T cells per grid for all conditions (B). Conditions are grouped by protein source (AB=human 10% AB serum; Plasma=2.5% human plasma; SR=5% serum replacement) and sub-divided into the presence (+) or absence (−) of IL-1β. See Table 2 for details of the different conditions. Bars are representative of experimental median. Dotted line represents median of experimental control (STD ISO).

FIG. 2: Graph showing % Vδ1 T cells of live cells (A) and total Vδ1 T cells per grid for all conditions (B). Conditions are grouped by protein source in the presence or absence of IL-1β. Bars are representative of experimental median. Dotted line represents median of experimental control.

FIG. 3: Graph showing results for viable cells per grid grouped by protein source and sub-divided into the presence or absence of IL-4. Bars are representative of experimental median. Dotted line represents median of experimental control.

FIG. 4: Graph showing results for % γδ T cells of live cells grouped by protein source and sub-divided into the presence or absence of IL-4. Bars are representative of experimental median. Dotted line represents median of experimental control.

FIG. 5: Graph showing results for total Vδ1 T cells per grid grouped by protein source and sub-divided into the presence or absence of IL-4. Bars are representative of experimental median. Dotted line represents median of experimental control.

FIG. 6: Graph showing results for % NKG2A expression (A) and % CD45RA expression (B) on Vδ1 T cells grouped by protein source and sub-divided into the presence or absence of IL-4. Bars are representative of experimental median. Dotted line represents median of experimental control.

FIG. 7: Graph showing results for % TIGIT expression on Vδ1 T cells grouped by protein source. Bars are representative of experimental median. Dotted line represents median of experimental control.

FIG. 8: Graph showing results for total Vδ1 T cells per grid grouped by protein source. Bars are representative of experimental median. Dotted line represents median of experimental control.

FIG. 9: Graph showing results for total viable cells per grid grouped by protein source. Bars are representative of experimental median. Dotted line represents median of experimental control.

FIG. 10: Graph showing results for the total TCR-negative cells grouped by protein source and sub-divided into the presence or absence of IFN-γ. Dotted line represents the median using previous isolation methods.

FIG. 11: Results of total γδ T cells per grid for the top conditions compared to the standard 2 cytokine isolation method.

FIG. 12: Graph showing the effect on fold expansion (A) and % γδ T cell expansion (B) using either freshly obtained isolated cells or isolated cells which have been frozen prior to expansion.

FIG. 13: A) Comparison of the total cell viability at the end of a 21 day isolation culture in the presence of IL-2, IL-4, IL-15 and IL-1β with or without the addition of IL-21 at 18.8 ng/ml. The dotted line represents the minimum acceptable viability. B) Graph showing the expansion after 14 days of culture (displayed as γδ T cells % of live) of γδ T cells isolated in the presence of IL-1β and IL-21 after thawing.

FIG. 14: Summary of isolation cultures either with “IL-21” (IL-2, IL-4, IL-15, IL-1β and IL-21) (IL-2, IL-4, IL-15 and IL-1β) or “No IL-21” (IL-2, IL-4, IL-15 and IL-1β) harvested after 19 or 21 days.

### DETAILED DESCRIPTION OF THE INVENTION

According to a first aspect of the invention, there is provided a method for the isolation of lymphocytes from a non-haematopoietic tissue sample comprising the steps of:
(i) culturing the non-haematopoietic tissue sample in the presence of Interleukin-1β (IL-1β); and
(ii) collecting a population of lymphocytes cultured from the non-haematopoietic tissue sample.

According to a further aspect of the invention, there is provided a method for the isolation of γδ T cells from a non-haematopoietic tissue sample comprising the steps of:
(i) culturing the non-haematopoietic tissue sample in the presence of IL-1β; and
(ii) collecting a population of γδ T cells cultured from the non-haematopoietic tissue sample.

The findings presented herein show a detailed design of experiments conducted to establish an optimal protocol for isolating tissue-resident γδ T cells from non-haematopoietic tissue samples. It was surprisingly found that all the conditions with the best γδ T cell yield contained IL-1β indicating that this cytokine was beneficial at yielding high levels of γδ T cells during the isolation process.

As used herein, “IL-1β” refers to native or recombinant IL-1β or a variant thereof that acts as an agonist for one or more IL-1 receptor (IL-1R) subunits (e.g. mutants, muteins, analogues, subunits, receptor complexes, fragments, isoforms, and peptidomimetics thereof). IL-1 is a pro-inflammatory cytokine that plays a major role in a wide range of diseases, including inflammatory diseases. It consists of two molecular species, IL-1a and IL-1β, which share only limited sequence identity but exert similar biological activities through binding to IL-1 receptor (type I and type II). Mature human IL-1β occurs as a 153 amino acid sequence after cleavage of 116 amino acids from the N-terminus in the precursor polypeptide by CASP1, as described in Andrei et al. (2004) PNAS 101(26): 9745-9750. An IL-1β mutein is a polypeptide wherein specific substitutions to the IL-1β protein have been made while retaining the ability to bind IL-1β. The IL-1β muteins can be characterized by amino acid insertions, deletions, substitutions and modifications at one or more sites in or at the other residues of the native IL-1β polypeptide chain. In accordance with this disclosure any such insertions, deletions, substitutions and modifications result in an IL-1β mutein that retains the IL-1R binding activity. Exemplary muteins can include substitutions of 1, 2, 3, 4, 5, 6, 7, 8, 9, 10 or more amino acids.

Nucleic acid encoding human IL-1β can be obtained by conventional procedures such as polymerase chain reaction (PCR). The amino acid sequence of human IL-1β (Gene ID 3553) is found in Genbank under accession locator NP_000567 or in UniProt under accession number P01584. The murine (Mus musculus) IL-1β amino acid sequence (Gene ID 16176) is found in Genbank under accession locator NP_032387 or in UniProt under accession number P10749.

IL-1β can also refer to IL-1β derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-1β variants are also encompassed by the invention. Examples of such variants are proteins that result from alternate mRNA splicing events or from proteolytic cleavage of the IL-1β protein, wherein the IL-1β binding property is retained.

Isolation Methods

References herein to “isolation” or “isolating” of cells, in particular of lymphocytes and/or γδ T cells, refer to methods or processes wherein cells are removed, separated, purified, enriched or otherwise taken out from a tissue or a pool of cells. It will be appreciated that such references include the terms “separated”, “removed”, “purified”, “enriched” and the like. Isolation of γδ T cells includes the isolation or separation of cells from an intact non-haematopoietic tissue sample or from the stromal cells of the non-haematopoietic tissue (e.g. fibroblasts or epithelial cells). Such isolation may alternatively or additionally comprise the isolation or separation of γδ T cells from other haematopoietic cells (e.g. αβ T cells or other lymphocytes). Isolation may be for a defined period of time, for example starting from the time the tissue explant or biopsy is placed in the isolation culture and ending when the cells are collected from culture, such as by centrifugation or other means for transferring the isolated cell population to expansion culture or used for other purposes, or the original tissue explant or biopsy is removed from the culture. The isolation step may be for at least about three days to about 45 days. In one embodiment, the isolation step is for at least about 10 days to at least 28 days. In a further embodiment, the isolation step is for at least 14 days to at least 21 days. The isolation step may therefore be for at least three days, four days, five days, six days, seven days, eight days, nine days, ten days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, about 35 days, about 40 days, or about 45 days. In one embodiment, the isolation step is for 19 days. In a yet further embodiment, the isolation step is for 21 days. It can be appreciated that although isolated cell proliferation may not be substantial during this isolation step, it is not necessarily absent. Indeed for someone skilled in the art it is recognized that isolated cells may also start to divide to generate a plurality of such cells within the isolation vessel containing the tissue and/or scaffold.

Thus, references herein to “isolated γδ T cells”, “isolated γδ T cell population”, “isolated population of γδ T cells”, “separated γδ T cells”, “separated γδ T cell population” or “separated population of γδ T cells” will be appreciated to refer to haematopoietic cells or a population of haematopoietic cells including γδ cells that have been isolated, separated, removed, purified or enriched from a non-haematopoietic tissue sample of origin such that the cells are out of substantial contact with non-haematopoietic cells or cells contained within the intact non-haematopoietic tissue. Likewise, references herein to an “isolated or separated population of Vδ1 T cells” refer to haematopoietic cells including Vδ1 T cells that have been isolated, separated, removed, purified or enriched from non-haematopoietic tissue sample of origin such that the cells are out of substantial contact with non-haematopoietic cells or cells contained within the intact non-haematopoietic tissue. Therefore, isolation or separation refers to the isolation, separation, removal, purification or enrichment of haematopoietic cells (e.g. γδ T cells or other lymphocytes) from non-haematopoietic cells (e.g. stromal cells, fibroblasts and/or epithelial cells).

Methods of isolation of γδ T cells as defined herein may comprise disruption of the tissue (e.g. mincing) followed by the separation of γδ T cells from other cell types. Preferably, methods of isolation of γδ T cells as defined herein may comprise “crawl-out” of γδ T cells and other cell types from an intact non-haematopoietic tissue sample or tissue matrix of the explant or biopsy, wherein the tissue resident lymphocytes physically separate from the tissue matrix without requiring the disruption of the tissue matrix. By maintaining the integrity of the tissue matrix, it has been found that the tissue resident lymphocytes preferentially egress from the tissue matrix with little or no egress of inhibitory cell types such as fibroblasts, which are retained in the explant or biopsy which can then be easily removed at the end of isolation. Thus, in some embodiments, the use of an intact non-haematopoietic tissue sample or tissue matrix leads to a low number of fibroblasts being released from the tissue into the culture. Such “crawl-out” methods utilising intact non-haematopoietic tissue or tissue matrix have the advantage of reducing the need for excess processing of the non-haematopoietic tissue sample or tissue matrix, maintain the structural integrity of the non-haematopoietic tissue or tissue matrix and may provide the unexpected advantage of delivering higher isolated cell yields.

Thus the methods of isolation of non-haematopoietic tissue derived lymphocytes as defined herein include methods for isolating non-haematopoietic tissue derived lymphocytes from an intact biopsy or explant of non-haematopoietic tissue. Such an intact biopsy or explant is one wherein the structural integrity of the biopsy or explant has not been deliberately disrupted within the perimeter of the excision removing the biopsy or explant from the tissue sample. Such an intact biopsy or explant will have the three-dimensional structure largely maintained except for minor disruption caused by handling. This intact biopsy or explant therefore has not been mechanically disrupted, such as by mincing or chopping, nor chemically enzymatically disrupted, for example. However, disrupted tissue may be used in the isolation methods of the present invention. In one embodiment, the isolated lymphocyte is an αβ T cell. In an alternative embodiment the isolated lymphocyte is a γδ T cell. In a further embodiment, the isolated lymphocyte is a TCR-negative cell (i.e. a cell which is negative for αβ TCR and γδ TCR expression). TCR negative cells are a good indicator of the presence of natural killer (NK) cells. Therefore, in another embodiment, the isolated lymphocyte is an NK cell. It can be appreciated that more than one type of lymphocyte may be isolated from the same isolation step.

Methods of isolation of γδ T cells utilising “crawl-out” or e.g. methods as defined herein, include the culturing of the cells and/or non-haematopoietic tissue sample in the presence of cytokines and/or chemokines sufficient to induce the isolation or separation of γδ T cells and/or other lymphocytes as defined herein.

In one embodiment, step (i) further comprises culturing the non-haematopoietic tissue sample in the presence of Interleukin-2 (IL-2). In another embodiment, step (i) further comprises culturing the non-haematopoietic tissue sample in the presence of Interleukin-15 (IL-15). In a yet further embodiment, step (i) further comprises culturing the non-haematopoietic tissue sample in the presence of IL-2 and IL-15.

As used herein, “IL-2” refers to native or recombinant IL-2 or a variant thereof that acts as an agonist for one or more IL-2 receptor (IL-2R) subunits (e.g. mutants, muteins, analogues, subunits, receptor complexes, fragments, isoforms, and peptidomimetics thereof). Such agents can support proliferation of an IL-2-dependent cell line, CTLL-2 (33; American Type Culture Collection (ATCC®) TIB 214). Mature human IL-2 occurs as a 133 amino acid sequence (less the signal peptide, consisting of an additional 20 N-terminal amino acids), as described in Fujita, et al. Cell 1986. 46.3:401-407. An IL-2 mutein is a polypeptide wherein specific substitutions to the Interleukin-2 protein have been made while retaining the ability to bind IL-2Rβ, such as those described in US 2014/0046026. The IL-2 muteins can be characterized by amino acid insertions, deletions, substitutions and modifications at one or more sites in or at the other residues of the native IL-2 polypeptide chain. In accordance with this disclosure any such insertions, deletions, substitutions and modifications result in an IL-2 mutein that retains the IL-2R8 binding activity. Exemplary muteins can include substitutions of 1, 2, 3, 4, 5, 6, 7, 8, 9, 10 or more amino acids.

Nucleic acid encoding human IL-2 can be obtained by conventional procedures such as PCR. The amino acid sequence of human IL-2 (Gene ID 3558) is found in Genbank under accession locator NP_000577.2 GI: 28178861. The murine (Mus musculus) IL-2 amino acid sequence (Gene ID 16183) is found in Genbank under accession locator NP_032392.1 GI: 7110653.

IL-2 can also refer to IL-2 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-2 variants are also encompassed by the invention. Examples of such variants are proteins that result from alternate mRNA splicing events or from proteolytic cleavage of the IL-2 protein, wherein the IL-2 binding property is retained. Alternate splicing of mRNA may yield a truncated but biologically active IL-2 protein. Variations attributable to proteolysis include, for example, differences in the N- or C-termini upon expression in different types of host cells, due to proteolytic removal of one or more terminal amino acids from the IL-2 protein (generally from 1-10 amino acids). In some embodiments, the terminus or interior of the protein can be modified to alter its physical properties, for example, with a chemical group such as polyethylene glycol (Yang, et al. Cancer 1995. 76: 687-694). In some embodiments, the terminus or interior of the protein can be modified with additional amino acids (Clark-Lewis, et al. PNAS 1993. 90:3574-3577).

As used herein, “IL-15” refers to native or recombinant IL-15 or a variant thereof that acts as an agonist for one or more IL-15 receptor (IL-15R) subunits (e.g. mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2. IL-15 was first reported by Grabstein, et al. (Grabstein, et al. Science 1994. 264.5161: 965-969) as a 114-amino acid mature protein. The term “IL-15” as used herein, means native or recombinant IL-15 and muteins, analogs, subunits thereof, or complexes thereof (e.g. receptor complexes, e.g. sushi peptides, as described in WO 2007/046006), and each of which can stimulate proliferation of CTLL-2 cells. In the CTLL-2 proliferation assays, supernatants of cells transfected with recombinantly expressed precursor and in-frame fusions of mature forms of IL-15 can induce CTLL-2 cell proliferation.

Human IL-15 can be obtained according to the procedures described by Grabstein, et al. (Grabstein, et al. Science 1994. 264.5161: 965-969) or by conventional procedures such as PCR. A deposit of human IL-15 cDNA was made with the ATCC® on Feb. 19, 1993 and assigned accession number 69245.

The amino acid sequence of human IL-15 (Gene ID 3600) is found in Genbank under accession locator NP000576.1 GI: 10835153 (isoform 1) and NP_751915.1 GI: 26787986 (isoform 2). The murine (Mus musculus) IL-15 amino acid sequence (Gene ID 16168) is found in Genbank under accession locator NP_001241676.1 GI: 363000984.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion or substitution. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-15 variants are also encompassed by the invention. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In one embodiment, the isolation of lymphocytes or γδ T cells according to the invention further comprises culturing the non-haematopoietic tissue sample in the presence of Interleukin-4 (IL-4). Thus, in a further embodiment, the non-haematopoietic tissue sample is cultured in the presence of IL-1β and IL-4.

As used herein, “IL-4” refers to native or recombinant IL-4 or a variant thereof that acts as an agonist for one or more IL-4 receptor (IL-4R) subunits (e.g. mutants, muteins, analogues, subunits, receptor complexes, fragments, isoforms, and peptidomimetics thereof). Such agents can support differentiation of naïve helper T cells (Th0 cells) to Th2 cells. Mature human IL-4 occurs as a 129 amino acid sequence (less the signal peptide, consisting of an additional 24 N-terminal amino acids). An IL-4 mutein is a polypeptide wherein specific substitutions to the Interleukin-4 protein have been made while retaining the ability to bind IL-4Rα, such as those described in U.S. Pat. No. 6,313,272. The IL-4 muteins can be characterized by amino acid insertions, deletions, substitutions and modifications at one or more sites in or at the other residues of the native IL-4 polypeptide chain. In accordance with this disclosure any such insertions, deletions, substitutions and modifications result in an IL-4 mutein that retains the IL-4Rα binding activity. Exemplary muteins can include substitutions of 1, 2, 3, 4, 5, 6, 7, 8, 9, 10 or more amino acids.

Nucleic acid encoding human IL-4 can be obtained by conventional procedures such as PCR. The amino acid sequence of human IL-4 (Gene ID 3565) is found in Genbank under accession locator NG_023252. The murine (Mus musculus) IL-4 amino acid sequence (Gene ID 16189) is found in Genbank under accession locator NC_000077.6.

IL-4 can also refer to IL-4 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-4 variants are also encompassed by the invention. Examples of such variants are proteins that result from alternate mRNA splicing events or from proteolytic cleavage of the IL-4 protein, wherein the IL-4 binding property is retained. Alternate splicing of mRNA may yield a truncated but biologically active IL-4 protein. Variations attributable to proteolysis include, for example, differences in the N- or C-termini upon expression in different types of host cells, due to proteolytic removal of one or more terminal amino acids from the IL-4 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In one embodiment, the isolation of lymphocytes or γδ T cells according to the invention further comprises culturing the non-haematopoietic tissue sample in the presence of Interferon-γ (IFN-γ). Thus, in a further embodiment, the non-haematopoietic tissue sample is cultured in the presence of IL-1β and IFN-γ.

As used herein, “IFN-γ” refers to native or recombinant IFN-γ or a variant thereof that acts as an agonist for one or more IFN-γ receptor (IFNGR) subunits (e.g. mutants, muteins, analogues, subunits, receptor complexes, fragments, isoforms, and peptidomimetics thereof). In particular, IFN-γ interacts with the heterodimeric receptor consisting of Interferon gamma receptor 1 (IFNGR1) and Interferon gamma receptor 2 (IFNGR2). Mature human IFN-γ occurs as a 143 amino acid sequence (less the signal peptide, consisting of an additional 23 N-terminal amino acids). An IFN-γ mutein is a polypeptide wherein specific substitutions to the IFN-γ protein have been made while retaining the ability to bind IFNGR, such as those described in U.S. Pat. No. 9,296,804. The IFN-γ muteins can be characterized by amino acid insertions, deletions, substitutions and modifications at one or more sites in or at the other residues of the native IFN-γ polypeptide chain. In accordance with this disclosure any such insertions, deletions, substitutions and modifications result in an IFN-γ mutein that retains the IFN-γR binding activity. Exemplary muteins can include substitutions of 1, 2, 3, 4, 5, 6, 7, 8, 9, 10 or more amino acids.

Nucleic acid encoding human IFN-γ can be obtained by conventional procedures such as PCR. The amino acid sequence of human IFN-γ (Gene ID 3458) is found in Genbank under accession locator NG_015840.1 or in UniProt under accession number P01579. The murine (Mus musculus) IFN-γ amino acid sequence (Gene ID 15978) is found in Genbank under accession locator NC_000076.6 or in UniProt under accession number P01580.

IFN-γ can also refer to IFN-γ derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IFN-γ variants are also encompassed by the invention. Examples of such variants are proteins that result from alternate mRNA splicing events or from proteolytic cleavage of the IFN-γ protein, wherein the IFN-γ binding property is retained.

In one embodiment, the isolation of lymphocytes or γδ T cells according to the invention further comprises culturing the non-haematopoietic tissue sample in the presence of Interleukin-21 (IL-21). Thus, in a further embodiment, the non-haematopoietic tissue sample is cultured in the presence of IL-1β and IL-21.

As used herein, “IL-21” refers to native or recombinant IL-21 or a variant thereof that acts as an agonist for one or more IL-21 receptor (IL-21R) subunits (e.g. mutants, muteins, analogues, subunits, receptor complexes, fragments, isoforms, and peptidomimetics thereof). Such agents can support proliferation of natural killer (NK) and cytotoxic (CD8⁺) T cells. Mature human IL-21 occurs as a 133 amino acid sequence (less the signal peptide, consisting of an additional 22 N-terminal amino acids). An IL-21 mutein is a polypeptide wherein specific substitutions to the Interleukin-21 protein have been made while retaining the ability to bind IL-21Rα, such as those described in U.S. Pat. No. 9,388,241. The IL-21 muteins can be characterized by amino acid insertions, deletions, substitutions and modifications at one or more sites in or at the other residues of the native IL-21 polypeptide chain. In accordance with this disclosure any such insertions, deletions, substitutions and modifications result in an IL-21 mutein that retains the IL-21R binding activity. Exemplary muteins can include substitutions of 1, 2, 3, 4, 5, 6, 7, 8, 9, 10 or more amino acids.

Nucleic acid encoding human IL-21 can be obtained by conventional procedures such as PCR. The amino acid sequence of human IL-21 (Gene ID 59067) is found in Genbank under accession locator NC_000004.12. The murine (Mus musculus) IL-21 amino acid sequence (Gene ID 60505) is found in Genbank under accession locator NC_000069.6.

IL-21 can also refer to IL-21 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-21 variants are also encompassed by the invention. Examples of such variants are proteins that result from alternate mRNA splicing events or from proteolytic cleavage of the IL-21 protein, wherein the IL-21 binding property is retained. Alternate splicing of mRNA may yield a truncated but biologically active IL-21 protein. Variations attributable to proteolysis include, for example, differences in the N- or C-termini upon expression in different types of host cells, due to proteolytic removal of one or more terminal amino acids from the IL-21 protein (generally from 1-10 amino acids). In some embodiments, the terminus of the protein can be modified to alter its physical properties, for example, with a chemical group such as polyethylene glycol (Yang, et al. Cancer 1995. 76:687-694). In some embodiments, the terminus or interior of the protein can be modified with additional amino acids (Clark-Lewis, et al. PNAS 1993. 90:3574-3577).

In alternative embodiments, the culturing is performed in the absence of IL-21. In alternative embodiments, the culturing is performed in the absence of IFN-γ. After statistical analysis of the results obtained from experiments described herein, it appears that the presence of IL-1β in the isolation culture may be more beneficial when at least IFN-γ is absent, such as when IFN-γ and IL-21 are absent. The results described herein further demonstrate that under certain conditions the presence of IL-1β in the isolation culture may also be beneficial when IL-21 is absent or when IL-21 is present at a concentration between 15 ng/mL and 25 ng/mL, in particular at a concentration of 18 to 20 ng/ml, such as 18, 19 or 20 ng/mL, for example 18.8 ng/mL.

In certain embodiments, the methods defined herein include IL-2 typically at a concentration of at least 10 IU/mL, such as at least 100 IU/mL (e.g., from 10 IU/mL to 1,000 IU/mL, from 20 IU/mL to 800 IU/mL, from 25 IU/mL to 750 IU/mL, from 30 IU/mL to 700 IU/mL, from 40 IU/mL to 600 IU/mL, from 50 IU/mL to 500 IU/mL, from 75 IU/mL to 250 IU/mL, or from 100 IU/mL to 200 IU/mL, e.g., from 10 IU/mL to 20 IU/mL, from 20 IU/mL to 30 IU/mL, from 30 IU/mL to 40 IU/mL, from 40 IU/mL to 50 IU/mL, from 50 IU/mL to 75 IU/mL, from 75 IU/mL to 100 IU/mL, from 100 IU/mL to 150 IU/mL, from 150 IU/mL to 200 IU/mL, from 200 IU/mL to 500 IU/mL, or from 500 IU/mL to 1,000 IU/mL). In certain embodiments, the methods defined herein include IL-2 typically at a concentration of less than 1,000 IU/mL, such as less than 500 IU/mL. In some embodiments, the methods include IL-2 at a concentration of about 100 IU/mL, such as 138 IU/mL.

In further embodiments, the methods defined herein include IL-15 typically at a concentration of at least 10 IU/mL, such as at least 100 IU/mL, in particular at least 500 IU/mL (e.g., from 10 IU/mL to 1,000 IU/mL, from 20 IU/mL to 900 IU/mL, from 25 IU/mL to 750 IU/mL, from 30 IU/mL to 600 IU/mL, from 40 IU/mL to 500 IU/mL, from 50 IU/mL to 400 IU/mL, from 75 IU/mL to 250 IU/mL, or from 100 IU/mL to 200 IU/mL, e.g., from 100 IU/mL to 900 IU/mL, from 200 IU/mL to 800 IU/mL, from 300 IU/mL to 700 IU/mL, from 400 IU/mL to 600 IU/mL, or from 500 IU/mL to 1,000 IU/mL). In certain embodiments, the methods defined herein include IL-15 typically at a concentration of less than 1,000 IU/mL, such as less than 700 IU/mL. In some embodiments, the methods include IL-15 at a concentration of about 600 IU/mL.

In some embodiments, the isolation of γδ T cells from the non-haematopoietic tissue sample includes culture in the presence of both IL-2 and IL-15, each at any of the concentrations listed above. In some cases, the concentration of IL-2 is about 138 IU/mL, and the concentration of IL-15 is 600 IU/mL.

In further embodiments, the methods defined herein include IL-21 typically at a concentration of at least 0.01 IU/mL, such as at least 0.1 IU/mL (e.g., from 0.01 IU/mL to 100 IU/mL, from 0.05 IU/mL to 50 IU/mL, from 0.1 IU/mL to 10 IU/mL, from 1 IU/mL to 5 IU/mL, e.g., from 0.01 IU/mL to 0.05 IU/mL, from 0.05 IU/mL to 0.1 IU/mL, from 0.1 IU/mL to 1 IU/mL, from 5 IU/mL to 10 IU/mL, from 10 IU/mL to 50 IU/mL, from 50 IU/mL to 100 IU/mL). In certain embodiments, the methods defined herein include IL-21 typically at a concentration of less than 10 IU/mL, such as less than 5 IU/mL. In some embodiments, the methods include IL-21 at a concentration of about 1 IU/mL, such as 1.05 IU/mL. In further embodiments, the methods include IL-21 at a concentration of 15 to 25 ng/mL. Thus, in one embodiment, the methods include IL-21 at a concentration between 15 ng/mL and 25 ng/mL. In a further embodiment, the methods include IL-21 at a concentration of 18 to 20 ng/ml, such as 18, 19 or 20 ng/mL, for example 18.8 ng/mL.

In further embodiments, the methods defined herein include IL-4 typically at a concentration of at least 1 IU/mL, such as at least 10 IU/mL (e.g., from 1 IU/mL to 1,000 IU/mL, from 5 IU/mL to 500 IU/mL, from 10 IU/mL to 250 IU/mL, from 50 IU/mL to 150 IU/mL, e.g., from 1 IU/mL to 5 IU/mL, from 5 IU/mL to 10 IU/mL, from 10 IU/mL to 50 IU/mL, from 50 IU/mL to 100 IU/mL, from 100 IU/mL to 150 IU/mL). In certain embodiments, the methods defined herein include IL-4 typically at a concentration of less than 500 IU/mL, such as less than 100 IU/mL. In some embodiments, the methods include IL-4 at a concentration of about 100 IU/mL, such as 95 IU/mL. In further embodiments, the methods include IL-4 at a concentration of about 30 IU/mL, such as 31.6 IU/mL.

Thus, in some embodiments the isolation of γδ T cells from the non-haematopoietic tissue sample includes culture in the presence of IL-2, IL-15, IL-4 and IL-21, each at any of the concentrations listed above. In further embodiments, the concentration of IL-2 is about 138 IU/mL, the concentration of IL-15 is 600 IU/mL, the concentration of IL-4 is 95 IU/mL and the concentration of IL-21 is between 15 ng/mL and 25 ng/mL, such as 18 to 20 ng/ml, such as 18, 19 or 20 ng/mL, for example 18.8 ng/mL. Thus, in a particular embodiment, the isolation of γδ T cells includes culture in the presence of IL-2 at a concentration of 138 IU/mL, IL-15 at a concentration of 600 IU/mL, IL-4 at a concentration of 95 IU/mL, IL-1β at a concentration of 4500 IU/mL, and optionally IL-21 at a concentration of 18.8 ng/mL.

References herein to “non-haematopoietic tissues” or “non-haematopoietic tissue sample” include skin (e.g. human skin) and gut (e.g. human gut). Non-haematopoietic tissue is a tissue other than blood, bone marrow, or thymus tissue. In one embodiment, the non-haematopoietic tissue sample is skin (e.g. human skin). In a further embodiment, the non-haematopoietic tissue sample is gut or gastrointestinal tract (e.g. human gut or human gastrointestinal tract). In some embodiments, the lymphocytes and/or γδ T cells are not obtained from particular types of samples of biological fluids, such as blood or synovial fluid. In some embodiments, the non-haematopoietic tissue sample from which the lymphocytes and/or γδ T cells are isolated according to the methods defined herein is skin (e.g. human skin), which can be obtained by methods known in the art. Alternatively, the methods of isolation of lymphocytes and/or γδ T cells provided herein can be applied to the gastrointestinal tract (e.g. colon or gut), mammary gland, lung, prostate, liver, spleen, pancreas, uterus, vagina and other cutaneous, mucosal or serous membranes. The lymphocytes and/or γδ T cells may also be resident in human cancer tissue samples, e.g. tumours of the breast or prostate. In some embodiments, the lymphocytes and/or γδ T cells may be from human cancer tissue samples (e.g. solid tumour tissues). In other embodiments, the lymphocytes and/or γδ T cells may be from non-haematopoietic tissue sample other than human cancer tissue (e.g. a tissue without a substantial number of tumour cells). For example, the lymphocytes and/or γδ T cells may be from a region of skin (e.g. healthy skin) separate from a nearby or adjacent cancer tissue. Thus, in some embodiments, the γδ T cells are not obtained from human cancer tissue. In further embodiments, the lymphocytes are not obtained from a human cancer tissue.

In one embodiment the non-haematopoietic tissue sample of the methods defined herein has been obtained from a human. In an alternative embodiment, the non-haematopoietic tissue sample of the methods defined herein has been obtained from a non-human animal subject.

Methods for obtaining such tissues are known in the art. Examples of such methods include scalpel explant or punch biopsy and may vary in size according to the method. In some embodiments, the non-haematopoietic tissue sample is obtained by punch biopsy.

In some embodiments of the present invention, the non-haematopoietic tissue sample is an intact biopsy. References herein to “intact” biopsy or “explant” include tissue and tissue sample that is not substantially disrupted, or not disrupted, such that the structural integrity of the biopsy or explant has not been deliberately disrupted within the perimeter of the excision removing the biopsy or explant from the tissue sample. Such an intact biopsy or explant will have the three dimensional structure largely maintained except for minor disruption caused by handling. This intact biopsy or explant therefore has not been mechanically disrupted, such as by mincing or chopping, nor chemically enzymatically disrupted, for example. An intact biopsy or intact tissue sample may comprise the whole tissue, the complete tissue, a portion of the tissue or all elements of said tissue. For example, in one embodiment the intact biopsy comprises all layers of the skin. In a further embodiment, the biopsy comprises the epidermal and dermal layers of the skin. It will be appreciated that in such embodiments wherein the biopsy is intact, separation and distinction between such layers is maintained. Thus, references herein to “intact” additionally include biopsies of full thickness of the non-haematopoietic tissue sample.

Thus, in one particular embodiment of the present invention, the non-haematopoietic tissue sample is not minced. In further embodiments, the intact biopsy is a punch biopsy. In a yet further embodiment, the intact biopsy is obtained by punch biopsy.

In one embodiment, the non-haematopoietic tissue sample is a punch biopsy. A punch biopsy may be of any shape, though is conveniently of circular cross-section and suitably is at least 1 mm in diameter. In yet further embodiments, the non-haematopoietic tissue sample comprises a punch biopsy at least 2 mm in diameter, such as at least 3 mm in diameter, at least 4 mm in diameter, at least 5 mm in diameter, at least 6 mm in diameter, at least 7 mm in diameter or at least 8 mm in diameter. In further embodiments, the non-haematopoietic tissue sample comprises a punch biopsy 8 mm or less in diameter, such as 7 mm or less in diameter, 6 mm or less in diameter, 5 mm or less in diameter or 3 mm or less in diameter. In one embodiment, the non-haematopoietic tissue sample comprises a punch biopsy of between 1 mm and 8 mm in diameter, such as between 2 mm and 4 mm in diameter. In a particular embodiment, the non-haematopoietic tissue sample comprises a punch biopsy of 3 mm in diameter.

In one embodiment, the biopsy is a skin biopsy and comprises the epidermal and dermal layers. In a further embodiment, the biopsy does not substantially comprise the subcutaneous fat. Thus, in one embodiment, the biopsy comprises epidermal and dermal layers and does not substantially comprise a layer of subcutaneous fat. In a further embodiment, the biopsy comprises no subcutaneous fat. Alternatively, the subcutaneous fat is not removed, therefore is present (or at least partially present) in the biopsy. Thus, in a yet further embodiment, the biopsy consists of epidermal and dermal layers. In one embodiment, the biopsy comprises the full thickness of the non-haematopoietic tissue sample.

Methods of the present invention comprise culturing non-haematopoietic tissue sample as defined herein. References herein to “culturing” include the addition of cells and/or a non-haematopoietic tissue sample, including isolated, separated, removed, purified or enriched cells from non-haematopoietic tissue sample, to media comprising growth factors and/or essential nutrients required and/or preferred by the cells and/or non-haematopoietic tissue sample. It will be appreciated that such culture conditions may be adapted according to the cells or cell population to be isolated from the non-haematopoietic tissue sample according to the invention or may be adapted according to the cells or cell population to be isolated and expanded from the non-haematopoietic tissue sample.

In certain embodiments, culturing of the non-haematopoietic tissue sample is for a duration of time sufficient for the isolation of γδ T cells from the non-haematopoietic tissue sample. In alternative embodiments, the culturing of non-haematopoietic tissue sample is for a duration of time sufficient for the isolation of lymphocytes other than γδ T cells from the non-haematopoietic tissue sample (e.g. αβ T cells and/or NK (natural killer) cells). In certain embodiments, the duration of culture according to the methods defined herein is at least 7 days. In certain embodiments, the duration of culture according to the methods defined herein is at least 14 days. In certain embodiments, the duration of culture according to the methods defined herein is less than 45 days, such as less than 40 days, such as less than 35 days, such as less than 30 days, such as less than 25 days. In a further embodiment, the duration of culture according to the methods defined herein is between 14 days and 35 days, such as between 14 days and 21 days. In a yet further embodiment, the duration of culture according to the methods defined herein is about 19 days, such as 19 days. In another embodiment, the duration of culture according to the methods defined herein is about 21 days, such as 21 days.

In particular embodiments of the present invention, the lymphocytes and/or γδ T cells isolated according to methods as defined herein are collected from the culture of non-haematopoietic tissue sample after culturing of the non-haematopoietic tissue sample. Collection of the lymphocytes and/or γδ T cells as defined herein may include the physical collection of lymphocytes and/or γδ T cells from the culture, isolation of the lymphocytes and/or γδ T cells from other lymphocytes (e.g. αβ T cells, γδ T cells and/or NK cells) or isolation and/or separation of the lymphocytes and/or γδ T cells from stromal cells (e.g. fibroblasts). In one embodiment, lymphocytes and/or γδ T cells are collected by mechanical means (e.g. pipetting). In a further embodiment, lymphocytes and/or γδ T cells are collected by means of magnetic separation and/or labelling. In a yet further embodiment, the lymphocytes and/or γδ T cells are collected by flow cytometric techniques such as FACS. Thus, in certain embodiments, the γδ T cells are collected by means of specific labelling the γδ T cells. In further embodiments, the lymphocytes are collected by means of specific labelling of the lymphocytes to distinguish them from other cells within the culture. It will be appreciated that such collection of lymphocytes and/or γδ T cells may include the physical removal from the culture of the non-haematopoietic tissue sample, transfer to a separate culture vessel or to separate or different culture conditions.

It will be appreciated that such collecting of lymphocytes and/or γδ T cells is performed after a duration of time sufficient to achieve an isolated population of lymphocytes and/or γδ T cells from the non-haematopoietic tissue sample. In certain embodiments, the lymphocytes and/or γδ T cells are collected after at least one week, at least 10 days, at least 11 days, at least 12 days, at least 13 days or at least 14 days of culturing of the non-haematopoietic tissue sample. Suitably, the lymphocytes and/or γδ T cells are collected after 40 days or less, such as 38 days or less, 36 days or less, 34 days or less, 32 days or less, 30 days or less, 28 days or less, 26 days or less or 24 days or less. In one embodiment, the lymphocytes and/or γδ T cells are collected after at least 14 days of culturing of the non-haematopoietic tissue sample. In a further embodiment, the lymphocytes and/or γδ T cells are collected after 14 to 21 days of culturing of the non-haematopoietic tissue sample. In a yet further embodiment, the lymphocytes and/or γδ T cells are collected after about 19 days of culturing, such as after 19 days. In another embodiment, the lymphocytes and/or γδ T cells are collected after about 21 days of culturing, such as after 21 days.

In certain embodiments of the present invention, the non-haematopoietic tissue sample is cultured in media which contains serum (e.g. human AB serum or fetal bovine serum (FBS)). In a further embodiment, the non-haematopoietic tissue is cultured in media containing 10% human AB serum. In another embodiment, the non-haematopoietic tissue is cultured in media containing 5% human AB serum. According to this embodiment, a serum replacement as defined hereinbelow may additionally be contained in the media. Thus, in a yet further embodiment the non-haematopoietic tissue is cultured in media containing 5% human AB serum and 5% serum replacement.

In certain embodiments of the present invention, the non-haematopoietic tissue sample is cultured in media which contains plasma (e.g. human plasma). In a further embodiment, the haematopoietic tissue is cultured in media containing 2.5% human plasma.

In an alternative embodiment of the present invention, the non-haematopoietic tissue sample is cultured in media which is substantially free of serum (e.g. serum-free media or media containing a serum-replacement (SR)). In a further embodiment, the haematopoietic tissue is cultured in media containing 5% serum replacement. Thus, in one embodiment, the non-haematopoietic tissue sample is cultured in serum-free media. Such serum free medium may also include serum replacement medium, where the serum replacement is based on chemically defined components to avoid the use of human or animal derived serum.

In one embodiment, the non-haematopoietic tissue sample is cultured in media which contains no animal-derived products.

In one embodiment, the methods as defined herein are performed in an isolation vessel. Reference to an “isolation vessel” refers to a vessel comprising the non-haematopoietic tissue sample for separation of the lymphocytes and/or γδ T cells, optionally further comprising a synthetic scaffold. It will be noted that the isolation vessel may be used just for the isolation method and not for the further expansion steps.

In one embodiment, the methods as defined herein are performed in a vessel (e.g. an isolation vessel) comprising a gas permeable material. Such materials are permeable to gases such as oxygen, carbon dioxide and/or nitrogen to allow gaseous exchange between the contents of the vessel and the surrounding atmosphere. It will be appreciated that references herein to “vessel” include culture dishes, culture plates, single-well dishes, multi-well dishes, multi-well plates, flasks, multi-layer flasks, bottles (such as roller bottles), bioreactors, bags, tubes and the like. Such vessels are known in the art for use in methods involving expansion of non-adherent cells and other lymphocytes. However, vessels comprising a gas permeable material also surprisingly find utility in the isolation of γδ T cells which are considered as usually being adherent. The use of such vessels for culturing was found to greatly increase the yield of isolated γδ T cells from non-haematopoietic tissue sample. Such vessels were also found to preferentially support γδ T cells and other lymphocytes over fibroblasts and other stromal cells (e.g. epithelial cells), including adherent cell-types. Thus, in one embodiment, the vessels comprising a gas permeable material as defined herein preferentially support γδ T cells and other lymphocytes (e.g. αβ T cells and/or NK cells). In a further embodiment, fibroblasts and/or other stromal cells (e.g. epithelial cells) are absent from cultures performed in vessels comprising a gas permeable material.

Such vessels comprising gas permeable materials may additionally comprise a gas permeable material that is non-porous. Thus, in one embodiment, the gas permeable material in non-porous. In some embodiments, the gas permeable material is a membrane film such as silicone, fluoroethylene polypropylene, polyolefin, or ethylene vinyl acetate copolymer. Furthermore, such vessels may comprise only a portion of gas permeable material, gas permeable membrane film or non-porous gas permeable material. Thus, according to a yet further embodiment, the vessel includes a top, a bottom and at least one sidewall, wherein at least part of the said vessel bottom comprises a gas permeable material that is in a substantially horizontal plane when said top is above said bottom. In one embodiment, the vessel includes a top, a bottom, and at least one sidewall, wherein at least a part of said bottom comprises the gas permeable material that is in a horizontal plane when said top is above said bottom. In a further embodiment, the vessel includes a top, a bottom and at least one sidewall, wherein the said at least one sidewall comprises a gas permeable material which may be in a vertical plane when said top is above said bottom, or may be a horizontal plane when said top is not above said bottom. It will be appreciated that in such embodiments, only a portion of said bottom or said side wall may comprise a gas permeable material. Alternatively, the entire of said bottom or entire of said sidewall may comprise a gas permeable material. In a yet further embodiment, said top of said vessel comprising a gas permeable material may be sealed, for example by utilisation of an O-ring. Such embodiments will be appreciated to prevent spillage or reduce evaporation of the vessel contents. Thus, in certain embodiments, the vessel comprises a liquid sealed container comprising a gas permeable material to allow gas exchange. In alternative embodiments, said top of said vessel comprising a gas permeable material is in the horizontal plane and above said bottom and is not sealed. Thus, in certain embodiments, said top is configured to allow gas exchange from the top of the vessel. In further embodiments, said bottom of the gas permeable container is configured to allow gas exchange from the bottom of the vessel. In a yet further embodiment, said vessel comprising a gas permeable material may be a liquid sealed container and further comprise inlet and outlet ports or tubes. Thus, in certain embodiments, the vessel comprising a gas permeable material includes a top, a bottom and optionally at least one sidewall, wherein at least a part of said top and said bottom comprise a gas permeable material and, if present, at least part of the at least one sidewall comprises a gas permeable material. Example vessels are described in WO2005035728 and U.S. Pat. No. 9,255,243 which are herein incorporated by reference. These vessels are also commercially available, such as the G-REX® cell culture devices provided by Wilson Wolf Manufacturing, such as the G-REX6 well-plate, G-REX24 well-plate and the G-REX10 vessel.

In one embodiment, the non-haematopoietic tissue sample is placed on a synthetic scaffold. As used herein, a “synthetic scaffold,” “scaffold,” and “grid” are used interchangeably and refer to a non-native three-dimensional structure suitable to support cell growth. A non-haematopoietic tissue sample may be either placed on or adhered to a synthetic scaffold to facilitate lymphocyte egress from the explant onto the scaffold. Synthetic scaffolds may be constructed from natural and/or synthetic materials such as polymers (e.g. natural or synthetic polymers, such as poly vinyl pyrrolidones, polymethylmethacrylate, methyl cellulose, polystyrene, polypropylene, polyurethane), ceramics (e.g. tricalcium phosphate, calcium aluminate, calcium hydroxyapatite), or metals (e.g. tantalum, titanium, platinum and metals in the same element group as platinum, niobium, hafnium, tungsten and combinations of alloys thereof). In one embodiment of the present invention, the synthetic scaffold is tantalum coated. Biological factors (e.g. collagens (such as collagen I or collagen II), fibronectins, laminins, integrins, angiogenic factors, anti-inflammatory factors, glycosaminoglycans, vitrogens, antibodies and fragments thereof, cytokines (e.g. IL-2, IL-15, IL-4, IL-21, IL-1β and combinations thereof, such as IL-2, IL-15, IL-4, IL-21, and combinations thereof) may be coated onto the scaffold surface, encapsulated within the scaffold material or added to the media to enhance cell adhesion, migration, survival, or proliferation, according to methods known in the art. This and other methods can be used to isolate lymphocytes from a number of other non-haematopoietic tissue types, e.g. skin, gut, prostate and breast.

In one embodiment, the non-haematopoietic tissue sample is placed on a synthetic scaffold inside the vessel used to isolate lymphocytes from the non-haematopoietic tissue sample. In a further embodiment, the synthetic scaffold is configured to facilitate lymphocyte and/or γδ T cell egress from the non-haematopoietic tissue sample to the bottom of the vessel. Such an embodiment has the advantage of allowing the isolation and/or separation of lymphocytes (e.g. γδ T cells, αβ T cells and/or NK cells) from the non-haematopoietic tissue sample and/or stromal cells (e.g. fibroblasts and/or epithelial cells). Furthermore, such embodiments allow the collection of lymphocytes (e.g. γδ T cells, αβ T cells and/or NK cells) from the non-haematopoietic tissue sample to the bottom of the culture vessel. In a particular embodiment, the synthetic scaffold is configured to facilitate the egress of γδ T cells from the non-haematopoietic tissue sample. In a further embodiment, the synthetic scaffold is configured to facilitate the egress of lymphocytes, such as αβ T cells and/or NK cells from the non-haematopoietic tissue sample.

Thus, in one aspect of the methods defined herein, the synthetic scaffold is configured to facilitate lymphocyte egress from the non-haematopoietic tissue sample to the bottom of the culture vessel. In a further aspect of the methods defined herein, synthetic scaffold is configured to facilitate γδ T cell egress from the non-haematopoietic tissue sample to the bottom of the vessel.

The methods of the present invention provide a total cell yield greater than previously described. In one embodiment, the total isolated cell number is at least 10⁶ cells/cm², at least 2×10⁶ cells/cm², at least 5×10⁶ cells/cm², at least 10×10⁶ cells/cm², at least 20×10⁶ cells/cm², at least 30×10⁶ cells/cm², at least 40×10⁶ cells/cm², at least 50×10⁶ cells/cm², at least 60×10⁶ cells/cm², at least 70×10⁶ cells/cm², at least 80×10⁶ cells/cm², at least 90×10⁶ cells/cm², at least 100×10⁶ cells/cm², at least 150×10⁶ cells/cm², at least 200×10⁶ cells/cm² of the tissue sample. In a specific embodiment, the total isolated cell number is at least at least 50×10⁶ cells/cm². In another embodiment, the total isolated cell number is at least at least 100×10⁶ cells/cm².

γδ T cells that are dominant in the blood are primarily Vδ2 T cells, while the γδ T cells that are dominant in the non-haematopoietic tissues are primarily Vδ1 T cells, such that Vδ1 T cells comprise about 70-80% of the non-haematopoietic tissue-resident γδ T cell population. However, some Vβ2 T cells are also found in non-haematopoietic tissues, e.g. in the gut, where they can comprise about 10-20% of γδ T cells. Some γδ T cells that are resident in non-haematopoietic tissues express neither Vδ1 nor Vβ2 TCR and have been referred to herein as double negative (DN) γδ T cells. These DN γδ T cells are likely to be mostly Vβ3-expressing with a minority of Vδ5-expressing T cells. Therefore, the γδ T cells that are ordinarily resident in non-haematopoietic tissues and that are isolated by the method of the invention are preferably non-Vβ2 T cells, e.g. Vδ1 T cells, with the inclusion of a smaller amount of DN γδ T cells.

Thus, in one preferred embodiment, the γδ T cells isolated by the methods defined herein comprise a population of Vδ1 T cells. In one embodiment, the γδ T cells isolated by the methods defined herein comprise a population of DN γδ T cells. In one embodiment, the γδ T cells isolated by the methods defined herein comprise a population of Vβ3 T cells. In one embodiment, the γδ T cells isolated by the methods defined herein comprise a population of Vβ5 T cells.

γδ T cells may also be defined by the type of γ chain that they express. In a further embodiment, the γδ T cells isolated by the methods defined herein comprise a population of Vγ4 T cells. Most often, Vγ4 T cells are obtained from gut tissue samples.

Methods of isolation provide an isolated population of γδ T cells that is greater in number than a reference population (e.g. at least 2-fold in number, at least 3-fold in number, at least 4-fold in number, at least 5-fold in number, at least 6-fold in number, at least 7-fold in number, at least 8-fold in number, at least 9-fold in number, at least 10-fold in number, at least 15-fold in number, at least 20-fold in number, at least 25-fold in number, at least 30-fold in number, at least 35-fold in number, at least 40-fold in number, at least 50-fold in number, at least 60-fold in number, at least 70-fold in number, at least 80-fold in number, at least 90-fold in number, at least 100-fold in number, at least 200-fold in number, at least 300-fold in number, at least 400-fold in number, at least 500-fold in number, at least 600-fold in number, at least 700-fold in number, at least 800-fold in number, at least 900-fold in number, at least 1,000-fold in number at least 5,000-fold in number, at least 10,000-fold in number).

In some embodiments, the population of γδ T cells isolated according to methods of the invention has a low proportion of cells expressing NKG2A. For example, the isolated population of γδ T cells may have a frequency of NKG2A+ cells of less than 40%, less than 35%, less than 30%, less than 20% or less than 10%. Alternatively, the isolated population of γδ T cells may have a frequency of NKG2A+ cells of about 40%, about 30%, about 20%, about 10%, about 9%, about 8%, about 7%, about 6%, about 5%, about 4%, about 3%, about 2% or about 1%. In certain embodiments, the isolated population of γδ T cells has a frequency of NKG2A+ cells of less than 10%. Thus, in one embodiment, the isolated population of γδ T cells has a frequency of NKG2A+ cells of about 8%. Thus, in one embodiment the isolated γδ T cells do not substantially express NKG2A.

In some embodiments, the population of Vδ1 T cells isolated according to methods of the invention has a low proportion of cells expressing NKG2A. For example, the isolated population of Vδ1 T cells may have a frequency of NKG2A+ cells of less than 40%, less than 35%, less than 30%, less than 20% or less than 10%. Alternatively, the isolated population of Vδ1 T cells may have a frequency of NKG2A+ cells of about 40%, about 30%, about 20%, about 10%, about 9%, about 8%, about 7%, about 6%, about 5%, about 4%, about 3%, about 2% or about 1%. In certain embodiments, the isolated population of Vδ1 T cells has a frequency of NKG2A+ cells of less than 10%. Thus, in one embodiment, the isolated population of Vδ1 cells has a frequency of NKG2A+ cells of about 9%. In another embodiment, the isolated population of Vδ1 cells has a frequency of NKG2A+ cells of about 7%. Thus, in one embodiment the isolated Vδ1 cells do not substantially express NKG2A. In one embodiment, less than 10% of the isolated population of Vδ1 T cells expresses NKG2A.

In some embodiments, the population of γδ T cells isolated according to methods of the invention has a low proportion of cells expressing CD45RA. CD45RA is a marker associated with terminal differentiation, therefore it is desirable to reduce the expression of this marker in the isolated cell population. For example, the isolated population of γδ T cells may have a frequency of CD45RA+ cells of less than 80%, less than 70%, less than 60%, less than 50%, less than 40% or less than 30%. Alternatively, the isolated population of γδ T cells may have a frequency of CD45RA+ cells of about 50%, about 40%, about 30%, about 20% or about 10%. In certain embodiments, the isolated population of γδ T cells has a frequency of CD45RA+ cells of less than 30%. Thus, in one embodiment, the isolated population of γδ T cells has a frequency of CD45RA+ cells of about 10%.

In some embodiments, the population of Vδ1 T cells isolated according to methods of the invention has a low proportion of cells expressing CD45RA. For example, the isolated population of Vδ1 T cells may have a frequency of CD45RA+ cells of less than 80%, less than 70%, less than 60%, less than 50%, less than 40% or less than 30%. Alternatively, the isolated population of Vδ1 T cells may have a frequency of CD45RA+ cells of about 50%, about 40%, about 30%, about 20% or about 10%. In certain embodiments, the isolated population of Vδ1 T cells has a frequency of CD45RA+ cells of less than 30%. Thus, in one embodiment, the isolated population of Vδ1 cells has a frequency of CD45RA+ cells of about 10%. In one embodiment, less than 80% of the isolated population of Vδ1 T cells expresses CD45RA, such as less than 30% of the isolated population of Vδ1 T cells expresses CD45RA.

Upon isolation from non-haematopoietic tissue (e.g. skin), the γδ T cells will generally be part of a larger population of lymphocytes containing, for example, αβ T cells, B cells, and natural killer (NK) cells. In some embodiments, 1%-10% of the isolated population of lymphocytes are γδ T cells (e.g. 1-10% of the isolated population of skin-derived lymphocytes are γδ T cells). In most cases, the γδ T cell population (e.g. skin-derived γδ T cell population) will include a large population of Vδ1 T cells. In some embodiments, 1-10% of the isolated population of lymphocytes (e.g. skin-derived lymphocytes) are Vδ1 T cells (e.g. Vδ1 T cells may represent over 50%, over 60%, over 70%, over 80%, or over 90% of the population of an isolated population γδ T cells). In some instances, less than 10% of the isolated population of γδ T cells are Vβ2 T cells (e.g. less than 10% of the isolated population of skin-derived γδ T cells are Vβ2 T cells).

Non-Vδ1 T cells or non-DN T cells, such as Vβ2 T cells, αβ T cells, B cells, or NK cells, may be removed from the isolated population of the γδ T cells (e.g. prior to, during, or after an expansion step).

Isolated γδ T cells (e.g. γδ T cells isolated from skin, e.g. Vδ1 T cells isolated from skin) have a distinct phenotype from corresponding haematopoietic tissue-derived cells (e.g. blood-derived γδ T cells and/or blood-derived Vβ2 T cells). For example, the isolated population of γδ T cells may express a higher level of CCR3, CCR4, CCR7, CCR8, or CD103 than a reference population, e.g. a TCR activated population of non-haematopoietic tissue-resident γδ T cells or a corresponding population of haematopoietic tissue-derived cells (e.g. blood-derived γδ T cells and/or blood-derived Vβ2 T cells). In some embodiments, the isolated population of γδ T cells includes at least 5%, 10%, 15%, 20%, 25%, 30%, 40%, 50%, 60%, 70%, 80%, 90% or more CCR3⁺ cells; at least 5%, 10%, 15%, 20%, 25%, 30%, 40%, 50%, 60%, 70%, 80%, 90% or more CCR4⁺ cells; at least 5%, 10%, 15%, 20%, 25%, 30%, 40%, 50%, 60%, 70%, 80%, 90% or more CCR7⁺ cells; at least 5%, 10%, 15%, 20%, 25%, 30%, 40%, 50%, 60%, 70%, 80%, 90% or more CCR8⁺ cells; and/or at least 5%, 10%, 15%, 20%, 25%, 30%, 40%, 50%, 60%, 70%, 80%, 90% or more CD103⁺ cells. The isolated population of γδ T cells may express one or more, two or more, three or more, four or more, five or more, or all six of CCR3, CCR4, CCR7, CCR8, or CD103.

The isolated population of non-haematopoietic tissue-derived γδ T cells (e.g. skin-derived γδ T cells and/or skin-derived Vδ1 T cells) can also be characterised by function. Functional assays known in the art can be performed to determine the functional differences between any non-haematopoietic tissue-derived cell of the invention (e.g. an isolated population of γδ T cells, skin-derived Vδ1 T cells, or an expanded population of γδ T cells and/or skin-derived Vδ1 T cells) and a reference cell (e.g. a TCR activated population of non-haematopoietic tissue-resident γδ T cells or a corresponding population of haematopoietic tissue-derived cells, e.g. blood-derived γδ T cells and/or blood-derived Vβ2 T cells). Such assays may include proliferation assays, cytotoxicity assays, binding assays, assays the measure persistence and/or location, etc.

Thus, in one aspect of the invention, the methods as defined herein for isolating a lymphocyte and/or γδ T cell population yields a population comprising a surface phenotype consistent with a non-exhausted lymphocyte and/or γδ T cell population.

Methods of the invention have also been shown to improve isolation of other lymphocytes, such as TCR-negative cells. Therefore, according to a further aspect of the invention, there is provided a method for the isolation of lymphocytes from a non-haematopoietic tissue sample comprising the steps of:
(i) culturing the non-haematopoietic tissue sample in media comprising serum replacement, human plasma and/or human AB serum; and
(ii) collecting a population of lymphocytes cultured from the non-haematopoietic tissue sample. Suitably the medium comprises serum replacement.

As shown in the Examples presented herein, protein source may have an impact on the numbers of TCR-negative cells isolated from the non-haematopoietic tissue sample. Thus, in one embodiment the isolated population of lymphocytes comprise a population of TCR-negative cells. It is known in the art that TCR-negative cell populations often contain high numbers of NK cells. The isolated population of TCR-negative cells may contain NK cells. In one embodiment, at least 50%, such as at least 60%, 70% or 80% of the isolated population of TCR-negative cells are NK cells.

The culture methods used in this aspect of the invention may contain the other conditions described herein. In one embodiment, step (i) further comprises culturing the non-haematopoietic tissue sample in the presence of IL-2 and IL-15.

In one embodiment, step (i) further comprises culturing the non-haematopoietic tissue sample in the presence of IL-4, IL-1β, IL-21 and/or IFN-γ. In another embodiment, step (i) further comprises culturing the non-haematopoietic tissue sample in the presence of IL-4 and/or IL-1β.

In one embodiment, the culturing is performed in the absence of IFN-γ. After statistical analysis of the results obtained from experiment described herein, it appears that the number of TCR-negative cells obtained during isolation may be increased when IFN-γ is absent. In one embodiment, the culturing is performed in the absence of IL-21. In an alternative embodiment, step (i) further comprises culturing the non-haematopoietic tissue sample in the presence of IL-21 at a concentration between 15 ng/mL and 25 ng/mL, such as 18 to 20 ng/ml, such as 18, 19 or 20 ng/mL. In a further embodiment, the culturing is performed in the presence of IL-21 at a concentration of 18.8 ng/mL.

In one embodiment, the method comprises freezing the isolated population of lymphocytes or γδ T cells. The cells may be frozen, for example, in Cryostor10 cell freezing solution. Many freezing solutions and parameters are known in the art and will be useful in this aspect of the invention. Frozen cells may be stored, for example between −80° C. and −200° C., optionally in liquid nitrogen (vapour phase), until required for use.

According to one aspect of the invention, there is provided an isolated population of lymphocytes (e.g. skin-derived αβ T cells and/or NK cells) obtained by any of the methods defined herein. In a further embodiment, the isolated population of lymphocytes is frozen.

According to one aspect of the invention, there is provided an isolated population of lymphocytes (e.g. skin-derived αβ T cells and/or NK cells) obtainable by any of the methods defined herein.

According to a further aspect of the invention, there is provided an isolated population of γδ T cells obtained by any of the methods defined herein. In a further embodiment, the isolated population of γδ T cells is frozen.

According to a further aspect of the invention, there is provided an isolated population of γδ T cells obtainable by any of the methods defined herein.

According to a further aspect of the invention, there is provided a method of isolation for γδ T cells from a non-haematopoietic tissue sample comprising the steps of:
(i) isolating a population of γδ T cells from the non-haematopoietic tissue sample; and
(ii) freezing the isolated population of γδ T cells.

Previous methods have involved freezing γδ T cells after expansion, however the current inventors have found that cells frozen after isolation are enriched and expand at least as well as fresh equivalents. Freezing post-isolation allows time to perform small-scale quality control expansion verification on donors before launching into large scale expansions which are huge resource cost and operator-intensive. It also enables the production of batches of cells from a single donor to be produced (i.e. in order to produce one batch of expanded cells, with the prospect of producing further batches from the same donor at later dates as required).

In this aspect of the invention, the method of isolation may comprise the methods described herein, or alternative isolation methods, such as culturing the non-haematopoietic tissue sample in the presence of IL-2, IL-4, IL-9, IL-15, IL-21 or combinations thereof, such as IL-2 and IL-15, in particular IL-2, IL-15 optionally in combination with IL-4 and/or IL-21. In one embodiment, the γδ T cells are isolated by culturing the non-haematopoietic tissue sample in the presence of IL-2 and IL-15, optionally in combination with IL-1β, IL-4 and/or IL-21. According to this embodiment, when present the IL-21 is at a concentration between 15 ng/mL and 25 ng/mL, such as 18, 19 or 20 ng/mL. Thus, in a further embodiment, when present the IL-21 is at a concentration of 18.8 ng/mL.

In one embodiment, isolating step (i) has a duration of at least 14 days. In other embodiments, isolating step (i) has a duration of less than 21 days. In yet further embodiments, isolating step (i) has a duration between 14 days and 35 days, such as about or between 19 days and 21 days. Thus, in one embodiment, isolating step (i) has a duration of about 19 days, such as 19 days. In another embodiment, isolating step (i) has a duration of about 21 days, such as 21 days.

The cells may be frozen in a suitable freezing solution, such as Cryostor10 cell freezing solution. Many freezing/cryopreservation solutions and parameters are known in the art and will be useful in this aspect of the invention. Suitable freezing solutions may contain DMSO and other suitable media supplements, such as human serum albumin, dextran, dextrose, NaCl, Hespan or PlasmaLyte A. Cells then are frozen to a temperature of about −80° C. to about −200° C., such as about −80° C. to about −135° C.

Cryopreservation may be accomplished by placing vials in a freezing container and then storing in a −80° C. freezer, for example for 1-3 days, followed by transfer to the vapor phase of a liquid nitrogen storage system. In an alternative embodiment, the isolated γδ T cells are frozen in a controlled rate freezer.

It will be understood that the frozen cells are suitable for long term storage, therefore the isolated cells can remain frozen for a duration of time before subsequent defrosting and expansion. Frozen cells may be stored, for example between −80° C. and −200° C., optionally in liquid nitrogen (vapour phase), until required for use.

After cryopreservation, cells may be thawed (i.e. defrosted), for example in a 37° C. water bath. The thawed cells may be subsequently used in an expansion method. Methods of expansion may comprise any of the methods described herein, or as described in the art, for example see WO2017072367 and WO2018202808.

Thus, the method may additionally comprise thawing the frozen population of γδ T cells. Furthermore, the method may comprise culturing the thawed population of γδ T cells for at least 5 days to produce an expanded population of γδ T cells.

Therefore, according to another aspect of the invention, there is provided a method of isolating and expanding γδ T cells from a non-haematopoietic tissue sample comprising the steps of:
(i) isolating γδ T cells from the non-haematopoietic tissue sample;
(ii) freezing the isolated γδ T cells;
(iii) thawing the γδ T cells; and
(iv) culturing the thawed γδ T cells for at least 5 days to produce an expanded population of γδ T cells.

In a particular embodiment, isolating step (i) comprises culturing the non-haematopoietic tissue sample in the presence of IL-21 at a concentration between 15 ng/mL and 25 ng/mL, such as 18 to 20 ng/ml, such as 18, 19 or 20 ng/mL for example 18.8 ng/mL, and IL-1β for a duration of about 19 days, such as 19 days. In another embodiment, isolating step (i) comprises culturing the non-haematopoietic tissue sample in the presence of IL-1β and the absence of IL-21 for a duration of about 19 days, such as 19 days, or about 21 days, such as 21 days. Data presented herein demonstrates that such isolating conditions yielded viable γδ T cells after thawing in step (iii) and the ability to expand during subsequent expansion culture, i.e. to effectively produce an expanded population of γδ T cells in step (iv).

According to another aspect of the invention, there is provided a frozen isolated γδ T cell population obtained by the method described herein (i.e. the frozen γδ T cells obtained at step (ii)).

According to another aspect of the invention, there is provided a frozen isolated γδ T cell population obtainable by the method described herein (i.e. the frozen γδ T cells obtained at step (ii)).

Expansion Methods

In certain embodiments, the invention features methods of expanding non-haematopoietic tissue-resident lymphocytes and/or γδ T cells (e.g. skin-derived αβ T cells, NK cells, γδ T cells and/or non-Vδ2 T cells, such as Vδ1 T cells and/or DN T cells). These methods may be carried out in vitro. In some embodiments, the γδ T cells are expanded from a population of γδ T cells that has been isolated from non-haematopoietic tissue sample according to methods defined herein. In general, non-haematopoietic tissue-resident γδ T cells are capable of spontaneously expanding upon removal of physical contact with stromal cells (e.g. skin fibroblasts). The methods defined herein can be used to induce such separation, resulting in de-repression of the γδ T cells to trigger expansion. In certain embodiments, lymphocytes (e.g. skin-derived αβ T cells and/or NK cells, gut-derived αβ T cells and/or NK cells) are expanded from a population of lymphocytes that has been isolated from non-haematopoietic tissue sample according to the methods defined herein.

As used herein, references to “expanded” or “expanded population of lymphocytes and/or γδ T cells” includes populations of cells which are larger or contain a larger number of cells than a non-expanded population. Such populations may be large in number, small in number or a mixed population with the expansion of a proportion or particular cell type within the population. It will be appreciated that the term “expansion step” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g. fold-increase or fold-expansion) are illustrative of an increase in the number or size of a population of cells or the number of cells and are indicative of the amount of expansion.

Thus, in one embodiment, the lymphocytes or γδ T cells isolated according to methods of the invention are expanded. In one embodiment, the isolated lymphocyte or γδ T cell population is frozen and then thawed prior to expansion.

Such expansion may comprise culturing the γδ T cells in the presence of IL-2, IL-15 and IL-21, optionally including IL-4. Alternatively, expansion may comprise culturing the γδ T cells in the presence of IL-9, IL-15 and IL-21, optionally including IL-4. It will be appreciated that any expansion step is performed for a duration of time effective to produce an expanded population of lymphocytes and/or γδ T cells. In one embodiment, a duration of time effective to produce an expanded population of lymphocytes and/or γδ T cells is at least 5 days. Thus, in one embodiment, expansion comprises culturing the γδ T cells in the presence of IL-2, IL-15 and IL-21 for at least 5 days in amounts effective to produce an expanded population of γδ T cells. In a further embodiment, expansion comprises culturing the γδ T cells in the presence of IL-2, IL-15, IL-21 and IL-4 for at least 5 days in amounts effective to produce an expanded population of γδ T cells. In a yet further embodiment, expansion comprises culturing the γδ T cells in the presence of IL-9, IL-15 and IL-21 for at least 5 days in amounts effective to produce an expanded population of γδ T cells. In one embodiment, expansion comprises culturing the γδ T cells in the presence of IL-9, IL-15, IL-21 and IL-4 for at least 5 days in amounts effective to produce an expanded population of γδ T cells.

In further embodiments, expansion comprises culturing the lymphocytes and/or γδ T cells for a duration (e.g. at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g. from 5 days to 40 days, from 7 days to 35 days, from 14 days to 28 days, or about 21 days) in an amount effective to produce an expanded population of γδ T cells. In some embodiments, the lymphocytes and/or γδ T cells are expanded in culture for a period of several hours (e.g. about 2, 3, 4, 5, 6, 7, 8, 9, 10, 15, 18, or 21 hours) to about 35 days (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days). In one embodiment, the lymphocytes and/or γδ T cells are expanded fora period of 14 to 21 days. Thus, including an isolation culture period (e.g. of 1 to 40 days, such as 14 to 21 days), the isolation and expansion steps, in some embodiments, can last between 28 and 56 days, or about 41 days.

In further embodiments, expansion comprises culturing the γδ T cells for at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g. from 5 days to 40 days, from 7 days to 35 days, from 14 days 28 days, or about 21 days. In one embodiment, the expansion step comprises culturing the γδ T cells for at least 10, 15 or 20 days to produce an expanded population. In one embodiment, the expansion step comprises culturing the γδ T cells between 5 and 25 days, such as between 14 and 21 days. In a further embodiment, the expansion step comprises culturing the γδ T cells for about 20 days.

In some embodiments, the typical amount of IL-2 effective to produce an expanded population of γδ T cells is from 1 IU/mL to 2,000 IU/mL (e.g. from 5 IU/mL to 1,000 IU/mL, from 10 IU/mL to 500 IU/mL, from 20 IU/mL to 400 IU/mL, from 50 IU/mL to 250 IU/mL, or about 100 IU/mL, e.g. from 5 IU/mL to 10 IU/mL, from 10 IU/mL to 20 IU/mL, from 20 IU/mL to 30 IU/mL, from 30 IU/mL to 40 IU/mL, from 40 IU/mL to 50 IU/mL, from 50 IU/mL to 60 IU/mL, from 60 IU/mL to 70 IU/mL, from 70 IU/mL to 80 IU/mL, from 80 IU/mL to 90 IU/mL, from 90 IU/mL to 100 IU/mL, from 100 IU/mL to 120 IU/mL, from 120 IU/mL to 140 IU/mL, from 140 IU/mL to 150 IU/mL, from 150 IU/mL to 175 IU/mL, from 175 IU/mL to 200 IU/mL, from 200 IU/mL to 300 IU/mL, from 300 IU/mL to 400 IU/mL, from 400 IU/mL to 500 IU/mL, from 500 IU/mL to 1,000 IU/mL, from 1,000 IU/mL to 1,500 IU/mL, from 1,500 IU/mL to 2,000 IU/mL, or greater). In some embodiments, the amount of IL-2 effective to produce an expanded population of γδ T cells is about 100 IU/mL.

In some embodiments, the typical amount of IL-15 effective to produce an expanded population of γδ T cells (e.g. skin-derived γδ T cells and/or non-Vδ2 T cells, such as Vδ1 T cells and/or DN T cells) is at least 0.1 ng/mL (e.g. from 0.1 ng/mL to 10,000 ng/mL, from 1.0 ng/mL to 1,000 ng/mL, from 5 ng/mL to 800 ng/mL, from 10 ng/mL to 750 ng/mL, from 20 ng/mL to 500 ng/mL, from 50 ng/mL to 400 ng/mL, or from 100 ng/mL to 250 ng/mL, e.g., from 0.1 ng/mL to 1.0 ng/mL, from 1.0 ng/mL to 5.0 ng/mL, from 5.0 ng/mL to 10 ng/mL, from 10 ng/mL to 20 ng/mL, from 20 ng/mL to 50 ng/mL, from 50 ng/mL to 100 ng/mL, from 100 ng/mL to 200 ng/mL, from 200 ng/mL to 500 ng/mL, or from 500 ng/mL to 1,000 ng/mL). In some embodiments, the amount of IL-15 effective to produce an expanded population of γδ T cells is about 10 ng/mL.

In some embodiments, the typical amount of IL-21 effective to produce an expanded population of γδ T cells (e.g. skin-derived γδ T cells and/or non-Vδ2 T cells, such as Vδ1 T cells and/or DN T cells) is at least 0.1 ng/mL, such as at least 1.0 ng/mL (e.g., from 0.1 ng/mL to 1,000 ng/mL, from 1.0 ng/mL to 100 ng/mL, from 1.0 ng/mL to 50 ng/mL, from 2 ng/mL to 50 ng/mL, from 3 ng/mL to 10 ng/mL, from 4 ng/mL to 8 ng/mL, from 5 ng/mL to 10 ng/mL, from 6 ng/mL to 8 ng/mL, e.g., from 0.1 ng/mL to 10 ng/mL, from 1.0 ng/mL to 5 ng/mL, from 1.0 ng/mL to 10 ng/mL, from 1.0 ng/mL to 20 ng/mL). In further embodiments, the amount of IL-21 is typically at a concentration of less than 200 ng/mL, such as 188.8 ng/mL. In other embodiments, the amount of IL-21 is typically at a concentration of less than 100 ng/mL, such as less 50 ng/mL, such as 37.5 ng/mL. In still other embodiments, the amount of IL-21 is typically at a concentration of 20 ng/mL or less, such as 18 to 20 ng/ml, such as 18, 19 or 20 ng/mL, for example 18.8 ng/mL. In some embodiments, the methods include IL-21 at a concentration of about 6 ng/mL, such as about 6.25 ng/mL.

In further embodiments, the methods defined herein include IL-4 typically at a concentration of at least 0.1 ng/mL, such as at least 10 ng/mL (e.g., from 0.1 ng/mL to 1,000 ng/mL, from 1.0 ng/mL to 100 ng/mL, from 1.0 ng/mL to 50 ng/mL, from 2 ng/mL to 50 ng/mL, from 3 ng/mL to 40 ng/mL, from 4 ng/mL to 30 ng/mL, from 5 ng/mL to 20 ng/mL, from 10 ng/mL to 20 ng/mL, e.g., from 0.1 ng/mL to 50 ng/mL, from 1.0 ng/mL to 25 ng/mL, from 5 ng/mL to 25 ng/mL). In further embodiments, the methods defined herein include IL-4 typically at a concentration of less than 100 ng/mL, such as less 50 ng/mL, in particular less than 20 ng/mL. In some embodiments, the methods include IL-4 at a concentration of about 15 ng/mL.

Substitution or addition of other factors in the expansion culture of non-haematopoietic tissue-resident lymphocytes and/or γδ T cells is also provided herein. For example, in some embodiments, any one or more factors selected from the group consisting of IL-4, IL-6, IL-7, IL-8, IL-9, IL-12, IL-18, IL-33, IGF-1, IL-1β, human platelet lysate (HPL), and stromal cell-derived factor-1 (SDF-1) is include in addition to, or in substitution of, any one of IL-2 and IL-15. Such additional or alternative factors for the expansion of lymphocytes such as αβ T cells or NK cells are known in the art. In one embodiment, such factors are used in the expansion which selectively promote the expansion of γδ T cells. In a further embodiment such factors are used in the expansion which selectively promote the expansion of lymphocytes such as αβ T cells and/or NK cells.

It will be understood that the amount of each of the above cytokines required to produce an expanded population of γδ T cells will depend of the concentrations of one or more of the other cytokines. For example, if the concentration of IL-2 is increased or decreased, the concentration of IL-15 may be accordingly decreased or increased, respectively. As noted above, the amount effective to produce an expanded population refers herein to composite effect of all factors on cell expansion.

Methods of expansion provide an expanded population of γδ T cells that is greater in number than a reference population. In some embodiments, the expanded population of γδ T cells is greater in number than the isolated population of γδ T cells prior to the expansion step (e.g. at least 2-fold in number, at least 3-fold in number, at least 4-fold in number, at least 5-fold in number, at least 6-fold in number, at least 7-fold in number, at least 8-fold in number, at least 9-fold in number, at least 10-fold in number, at least 15-fold in number, at least 20-fold in number, at least 25-fold in number, at least 30-fold in number, at least 35-fold in number, at least 40-fold in number, at least 50-fold in number, at least 60-fold in number, at least 70-fold in number, at least 80-fold in number, at least 90-fold in number, at least 100-fold in number, at least 200-fold in number, at least 300-fold in number, at least 400-fold in number, at least 500-fold in number, at least 600-fold in number, at least 700-fold in number, at least 800-fold in number, at least 900-fold in number, at least 1,000-fold in number at least 5,000-fold in number, at least 10,000-fold in number, or more relative to the isolated population of γδ T cells prior to the expansion step).

In one embodiment, the expansion step comprises culturing the isolated γδ T cells in the absence of substantial stromal cell contact. In a further embodiment, the expansion step comprises culturing the isolated γδ T cells in the absence of substantial fibroblast cell contact.

In further embodiments, the expansion step further comprises culturing the isolated γδ T cells in the presence of IL-4. Therefore, in one embodiment, expansion comprises culturing the isolated γδ T cells in the presence of IL-2, IL-15, IL-4 and IL-21. Alternatively, expansion may comprise culturing the isolated γδ T cells in the presence of IL-9, IL-15, IL-4 and IL-21.

It will be appreciated that methods of expansion defined herein also apply to the expansion of other lymphocytes (e.g. αβ T cells and/or NK cells). In such embodiments, the expansion step comprises culturing the isolated lymphocytes in the presence of the relevant growth factors and/or nutrients (e.g. cytokines and/or chemokines) to produce an expanded population of lymphocytes (e.g. αβ T cells and/or NK cells).

In one embodiment, the methods of expanding a population of γδ T cells as defined herein comprise culturing the γδ T cells or other lymphocytes in media containing serum or plasma. In alternative embodiments, the methods of expanding a population of γδ T cells or other lymphocytes as defined herein comprise culturing the γδ T cells in serum-free medium. In a further embodiment, the methods of expanding a population of γδ T cells or other lymphocytes as defined herein comprise culturing the γδ T cells in medium containing serum-replacement.

in some embodiments, no substantial TCR pathway activation is present during the expansion step (e.g. no exogenous TCR pathway activators are included in the culture). In one embodiment, the expansion step comprises the absence of exogenous TCR pathway agonists. Further, provided herein are methods of expanding γδ T cells isolated according to the methods defined herein, wherein said expansion methods do not involve contact with feeder cells, tumour cells, and/or antigen-presenting cells. Thus, in a further embodiment of the methods defined herein, the expansion of γδ T cells comprises culturing the γδ T cells in the absence of substantial stromal cell contact.

Also provided is a means to produce large populations of non-haematopoietic tissue-derived γδ T cells (e.g. skin-derived γδ T cells and/or non-Vδ2 T cells, such as Vδ1 T cells and/or DN T cells) at high rates (e.g. by removing stromal cell contact and/or TCR stimulation, or by culturing in the presence of an effective amount of factors). In some embodiments, the expansion step described herein expands the γδ T cells at a low population doubling time, which is given by the following equation:

$$DoublingTime = \frac{duration * log ( 2 )}{log ( FinalConcentration ) - log ( InitialConcentration )}$$

Given the information provided herein, a skilled artisan will recognize that the invention provides methods of expanding non-haematopoietic tissue-derived γδ T cells (e.g. skin-derived γδ T cells and/or non-Vδ2 T cells, such as Vδ1 T cells and/or DN T cells) at a population doubling time of less than 5 days (e.g. less than 4.5 days, less than 4.0 days, less than 3.9 days, less than 3.8 days, less than 3.7 days, less than 3.6 days, less than 3.5 days, less than 3.4 days, less than 3.3 days, less than 3.2 days, less than 3.1 days, less than 3.0 days, less than 2.9 days, less than 2.8 days, less than 2.7 days, less than 2.6 days, less than 2.5 days, less than 2.4 days, less than 2.3 days, less than 2.2 days, less than 2.1 days, less than 2.0 days, less than 46 hours, less than 42 hours, less than 38 hours, less than 35 hours, less than 32 hours).

In some embodiments, within 7 days of culture, the expanded population of γδ T cells (e.g. the expanded population of Vδ1 T cells and/or DN T cells) comprises at least 10-fold the number of γδ T cells relative to the isolated population of γδ T cells prior to expansion (e.g. at least 20-fold, at least 30-fold, at least 40-fold, at least 50-fold, at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, at least 1,000-fold, at least 2,000-fold, at least 3,000-fold, at least 4,000-fold, at least 5,000-fold, at least 6,000-fold, at least 7,000-fold, or at least 8,000-fold the number of γδ T cells relative to the isolated population of γδ T cells prior to expansion). In some embodiments, within 14 days of culture, the expanded population of γδ T cells (e.g. the expanded population of Vδ1 T cells and/or DN T cells) comprises at least 20-fold the number of γδ T cells relative to the isolated population of γδ T cells prior to expansion (e.g. at least 30-fold, at least 40-fold, at least 50-fold, at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, at least 1,000-fold, at least 2,000-fold, at least 3,000-fold, at least 4,000-fold, at least 5,000-fold, at least 6,000-fold, at least 7,000-fold, at least 8,000-fold, at least 9,000-fold, or at least 10,000-fold the number of γδ T cells relative to the isolated population of γδ T cells prior to expansion). In some embodiments, within 21 days of culture, the expanded population of γδ T cells (e.g. the expanded population of Vδ1 T cells and/or DN T cells) comprises at least 50-fold the number of γδ T cells relative to the isolated population of γδ T cells prior to expansion (e.g. at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, at least 1,000-fold, at least 2,000-fold, at least 3,000-fold, at least 4,000-fold, at least 5,000-fold, at least 6,000-fold, at least 7,000-fold, at least 8,000-fold, at least 9,000-fold, or least 10,000-fold the number of γδ T cells relative to the isolated population of γδ T cells prior to expansion). In some embodiments, within 28 days of culture, the expanded population of γδ T cells (e.g. the expanded population of Vδ1 T cells and/or DN T cells) comprises at least 100-fold the number of γδ T cells relative to the isolated population of γδ T cells prior to expansion (e.g. at least 110-fold, at least 120-fold, at least 130-fold, at least 140-fold, at least 150-fold, at least 200-fold, at least 300-fold, at least 400-fold, at least 500-fold, at least 600-fold, at least 700-fold, at least 800-fold, at least 900-fold, at least 1,000-fold, at least 2,000-fold, at least 3,000-fold, at least 4,000-fold, at least 5,000-fold, at least 6,000-fold, at least 7,000-fold, at least 8,000-fold, at least 9,000-fold, at least 10,000-fold, at least 12,000-fold, or at least 15,000-fold the number of γδT cells relative to the isolated population of γδ T cells prior to expansion).

Non-haematopoietic tissue-derived γδ T cells (e.g. skin-derived γδ T cells and/or non-Vδ2 T cells, such as Vδ1 T cells and/or DN T cells) expanded by the methods provided herein can have a phenotype well-suited for anti-tumor efficacy. In some embodiments, the expanded population of γδ T cells (e.g. skin-derived Vδ1 T cells) has a greater mean expression of CD27 than a reference population (e.g. the isolated population of γδ T cells prior to the expansion step). In some embodiments, the expanded population of γδ T cells has a mean expression of CD27 that is at least 2-fold relative to the isolated population of γδ T cells (e.g. at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 150-fold, at least 200-fold, at least 300-fold, at least 400-fold, at least 500-fold, at least 600-fold, at least 700-fold, at least 800-fold, at least 900-fold, at least 1,000-fold, at least 5,000-fold, at least 10,000-fold, at least 20,000-fold, or more, relative to the isolated population of γδ T cells).

A distinct portion of the expanded population of γδ T cells (e.g. skin-derived γδ T cells and/or non-Vδ2 T cells, such as Vδ1 T cells and/or DN T cells) may upregulate CD27, while another portion is CD27^{low} or CD27^{negative}. In this case, the frequency of CD27^{positive} cells in the expanded population relative to the isolated population of γδ T cells may be greater. For example, the expanded population of γδ T cells may have at least a 5% greater frequency of CD27^{positive} cells relative to that of the isolated population of γδ T cells prior to expansion (e.g. at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or up to 100% greater frequency of CD27^{positive} cells relative to that of the isolated population of γδ T cells prior to expansion). In some embodiments, the number of CD27^{positive} cells in the expanded population relative to the isolated population of γδ T cells may be increased. For example, the expanded population of γδ T cells may have at least 2-fold the number of CD27^{positive} cells relative to the isolated population of γδ T cells prior to expansion. The expanded population of γδ T cells may have a frequency of CD27+ cells of greater than 10%, greater than 20%, greater than 30%, greater than 40%, greater than 50%, greater than 60%, greater than 70%, greater than 80% or greater than 90%. Alternatively, the expanded population of γδ T cells may have a frequency of CD27+ cells of about 10%, about 20%, about 30%, about 40%, about 50%, about 60%, about 70%, about 80% or about 90%. In certain embodiments, the expanded population of γδ T cells has a frequency of CD27+ cells of greater than 50%.

Methods of expansion as provided herein, in some embodiments, yield an expanded population non-haematopoietic tissue-derived γδ T cells (e.g. skin-derived γδ T cells and/or non-Vδ2 T cells, such as Vδ1 T cells and/or DN T cells) having a low expression of TIGIT, relative to a reference population (e.g. the isolated population of γδ T cells prior to the expansion step). In some embodiments, the expanded population of γδ T cells has a lower mean expression of TIGIT than a reference population (e.g. the isolated population of γδ T cells prior to the expansion step). In some embodiments, the expanded population of γδ T cells has a mean expression of TIGIT that is at least 10% less than the isolated population of γδ T cells (e.g. at least 20% less, at least 30% less, at least 40% less, at least 50% less, at least 60% less, at least 70% less, at least 80% less, at least 90% less, or up to 100% less than the isolated population of γδ T cells). The expanded population of γδ T cells may have a frequency of TIGIT+ cells of less than 90%, less than 80%, less than 70%, less than 60%, less than 50%, less than 40%, less than 30%, less than 20% or less than 10%. Alternatively, the expanded population of γδ T cells may have a frequency of TIGIT+ cells of about 90%, about 80%, about 70%, about 60%, about 50%, about 40%, about 30%, about 20% or about 10%. In certain embodiments, the isolated population of γδ T cells has a frequency of TIGIT+ cells of less than 80%.

In some embodiments, the expanded population of γδ T cells (e.g. skin-derived γδ T cells or non-Vδ2 T cells, such as Vδ1 T cells and/or DN T cells) has a high number or frequency of CD27⁺ cells and a low frequency of TIGIT+ cells. In some embodiments, the expanded population of γδ T cells has a high frequency of CD27⁺TIGIT⁻ cells relative to a reference population (e.g. relative to an isolated population of γδ T cells prior to expansion). For instance, the expanded population of γδT cells may have at least a 5% greater frequency of CD27⁺ TIGIT⁻ cells relative to that of the isolated population of γδ T cells prior to expansion (e.g. at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or up to 100% greater frequency of CD27⁺ TIGIT⁻ cells relative to that of the isolated population of γδ T cells prior to expansion). In some embodiments, the number of CD27⁺ TIGIT⁻ cells in the expanded population relative to the isolated population of γδ T cells may be increased. For example, the expanded population of γδT cells may have at least 2-fold the number of CD27⁺ TIGIT⁻ cells relative to the isolated population of γδ T cells prior to expansion (e.g. at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or up to 100% greater frequency of CD27⁺TIGIT⁻ cells relative to that of the isolated population of γδ T cells prior to expansion).

In some instances, the mean expression of TIGIT on a population of CD27⁺ γδ T cells in an expanded population of γδ T cells (e.g. skin-derived γδ T cells and/or non-Vδ2 T cells, such as Vδ1 T cells and/or DN T cells) is low relative to a reference population. In some embodiments, the expanded population of CD27⁺ γδ T cells has a lower mean expression of TIGIT than a reference population (e.g. the isolated population of CD27⁺ γδ T cells prior to the expansion step). In some embodiments, the expanded population of CD27⁺ γδ T cells has a mean expression of TIGIT that is at least 10% less than the isolated population of CD27⁺ γδ T cells (e.g. at least 20% less, at least 30% less, at least 40% less, at least 50% less, at least 60% less, at least 70% less, at least 80% less, at least 90% less, or up to 100% less than the isolated population of CD27⁺ γδ T cells).

Additionally or alternatively, the median expression of CD27 on a population of TIGIT⁻ γδ T cells in an expanded population of γδ T cells (e.g. skin-derived γδ T cells and/or non-Vδ2 T cells, such as Vδ1 T cells and/or DN T cells) is high relative to a reference population. For example, the expanded population of TIGIT⁻ γδ T cells may have at least a 5% greater frequency of CD27⁺ cells relative to that of the isolated population of TIGIT⁻ γδ T cells prior to expansion (e.g. at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or up to 100% greater frequency of CD27⁺ cells relative to that of the isolated population of TIGIT⁻ γδ T cells prior to expansion). In some embodiments, the number of CD27⁺ cells in the expanded population relative to the isolated population of TIGIT⁻ γδ T cells may be increased. For example, the expanded population of TIGIT⁻ γδ T cells may have at least 2-fold the number of CD27⁺ cells relative to the isolated population of TIGIT⁻ γδ T cells prior to expansion (e.g. at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or up to 100% greater frequency of CD27⁺ cells relative to that of the isolated population of TIGIT⁻ γδ T cells prior to expansion).

An increase or decrease in expression of other markers can be additionally or alternatively used to characterize one or more expanded populations of non-haematopoietic tissue-derived γδ T cells (e.g. skin-derived γδ T cells and/or non-Vδ2 T cells, such as Vδ1 T cells and/or DN T cells), including CD124, CD215, CD360, CTLA4, CD1b, BTLA, CD39, CD45RA, Fas Ligand, CD25, ICAM-1, CD31, KLRG1, CD30, CD2, NKp44, NKp46, ICAM-2, CD70, CD28, CD103, NKp30, LAG3, CCR4, CD69, PD-1, and CD64. In some instances, the expanded population of γδ T cells (e.g. skin-derived γδ T cells and/or non-Vδ2 T cells, such as Vδ1 T cells and/or DN T cells) has a greater mean expression of one or more of the markers selected from the group consisting of CD124, CD215, CD360, CTLA4, CD1b, BTLA, CD39, CD45RA, Fas Ligand, CD25, ICAM-1, CD31, KLRG1, CD30, and CD2, relative to the isolated population of γδ T cells, e.g. prior to expansion. Additionally or alternatively, the expanded population of γδ T cells may have a greater frequency of cells expressing one or more of the markers selected from the group consisting of CD124, CD215, CD360, CTLA4, CD1b, BTLA, CD39, CD45RA, Fas Ligand, CD25, ICAM-1, CD31, KLRG1, CD30, and CD2, relative to the isolated population of γδ T cells. In some embodiments, the expanded population of γδ T cells has a lower mean expression of one or more of the markers selected from the group consisting of NKp44, NKp46, ICAM-2, CD70, CD28, CD103, NKp30, LAG3, CCR4, CD69, PD-1, and CD64, relative to the isolated population of γδ T cells. The expanded population may similarly have a lower frequency of cells expressing one or more of the markers selected from the group consisting of NKp44, NKp46, ICAM-2, CD70, CD28, CD103, NKp30, LAG3, CCR4, CD69, PD-1, and CD64, relative to the isolated population of γδ T cells.

Numerous basal culture media suitable for use in the culturing and/or proliferation of γδ T cells are available, in particular medium, such as AIM-V, Iscoves medium and RPMI-1640 (Life Technologies). The medium may be supplemented with other media factors as defined herein, such as serum, serum proteins and selective agents, such as antibiotics. For example, in some embodiments, RPMI-1640 medium containing 2 mM glutamine, 10% FBS, 10 mM HEPES, pH 7.2, 1% penicillin-streptomycin, sodium pyruvate (1 mM; Life Technologies), non-essential amino acids (e.g. 100 μM Gly, Ala, Asn, Asp, Glu, Pro and Ser; 1× MEM non-essential amino acids (Life Technologies)), and 10 μl/L β-mercaptoethanol. In an alternative embodiment, AIM-V medium may be supplemented with CTS Immune serum replacement and amphotericin B. In certain embodiments as defined herein, the media may be further supplemented with IL-2 and IL-15. Conveniently, cells are cultured at 37° C. in a humidified atmosphere containing 5% CO₂ in a suitable culture medium during isolation and/or expansion.

According to a further aspect of the invention there is provided a method for the isolation and expansion of lymphocytes from a non-haematopoietic tissue sample comprising the steps of:
(i) isolating a population of lymphocytes from the non-haematopoietic tissue sample according to the method defined herein; and
(ii) further culturing said population of lymphocytes (such as for at least 5 days) to produce an expanded population of lymphocytes.

In one embodiment, the lymphocytes comprise αβ T cells. Therefore, according to a further aspect of the invention there is provided a method for the isolation and expansion of αβ T cells from a non-haematopoietic tissue sample comprising the steps of:
(i) isolating a population of αβ T cells from the non-haematopoietic tissue sample according to the method defined herein; and
(ii) further culturing said population of αβ T cells (such as for at least 5 days) to produce an expanded population of αβ T cells.

Culturing in step (ii) may be by selective expansion, such as by choosing culturing conditions where αβ T cells are preferentially expanded over other cells types present in the isolated population in step (i). Alternatively, the expansion conditions are not selective and culturing in step (ii) may be followed by depletion of non-target cells (e.g. cells other than αβ T cells). Alternatively, the expansion conditions are not selective and depletion of non-target cells (e.g. cells other than αβ T cells) occurs prior to culturing in step (ii). It is noted that the objective of these embodiments is to expand the total number of αβ T cells while also increasing their proportion in the population.

In one embodiment, the lymphocytes comprise NK cells. Therefore, according to a further aspect of the invention there is provided a method for the isolation and expansion of NK cells from a non-haematopoietic tissue sample comprising the steps of:
(i) isolating a population of NK cells from the non-haematopoietic tissue sample according to the method defined herein; and
(ii) further culturing said population of NK cells (such as for at least 5 days) to produce an expanded population of NK cells.

Culturing in step (ii) may be by selective expansion, such as by choosing culturing conditions where NK cells are preferentially expanded over other cells types present in the isolated population in step (i). Alternatively, the expansion conditions are not selective and culturing in step (ii) may be followed by depletion of non-target cells (e.g. cells other than NK cells). Alternatively, the expansion conditions are not selective and depletion of non-target cells (e.g. cells other than NK cells) occurs prior to culturing in step (ii). It is noted that the objective of these embodiments is to expand the total number of NK cells while also increasing their proportion in the population.

According to a further aspect of the invention there is provided a method for the isolation and expansion of γδ T cells from a non-haematopoietic tissue sample comprising the steps of:
(i) isolating a population of γδ T cells from the non-haematopoietic tissue sample according to the method defined herein; and
(ii) further culturing said population of γδ T cells (such as for at least 5 days) to produce an expanded population of γδ T cells.

Culturing in step (ii) may be by selective expansion, such as by choosing culturing conditions where γδ T cells are preferentially expanded over other cells types present in the isolated population in step (i). Alternatively, the expansion conditions are not selective and culturing in step (ii) may be followed by depletion of non-target cells (e.g. cells other than γδ T cells). Alternatively, the expansion conditions are not selective and depletion of non-target cells (e.g. cells other than γδ T cells) occurs prior to culturing in step (ii). It is noted that the objective of these embodiments is to expand the total number of γδ T cells while also increasing their proportion in the population.

In one embodiment, the isolated lymphocyte or γδ T cell population is frozen and then thawed prior to step (ii). It has surprisingly been found that frozen populations of isolated cells enrich and expand at least as well as fresh equivalents. In particular, the data presented herein demonstrates that isolated γδ T cells with good viability after thawing and the ability to expand well during subsequent expansion culture, i.e. to effectively produce an expanded population of γδ T cells in step (ii), are achieved wherein isolating step (i) comprises culturing the non-haematopoietic tissue sample in the presence of IL-21 at a concentration between 15 ng/mL and 25 ng/mL, such as 18 to 20 ng/ml, such as 18, 19 or 20 ng/mL, for example 18.8 ng/mL, and IL-1β fora duration of about 19 days, such as 19 days, or alternatively in the presence of IL-1β and the absence of IL-21 fora duration of about 19 days, such as 19 days, or about 21 days, such as 21 days.

In one embodiment, the lymphocytes comprise γδ T cells. Therefore, according to a further aspect of the invention, there is provided a method for the isolation and expansion of γδ T cells from a non-haematopoietic tissue sample comprising the steps of:
(i) isolating a population of γδ T cells from a non-haematopoietic tissue sample according to the method defined herein; and
(ii) culturing said population of γδ T cells in the presence of:
   (a) IL-2 or IL-9;
   (b) IL15; and
   (c) IL-21
for at least 5 days in amounts effective to produce an expanded population of γδ T cells.

In certain embodiments of this aspect of the invention, culturing said population of γδ T cells further comprises the presence of IL-4. Thus, in a further aspect of the invention, there is provided a method for the isolation and expansion of γδ T cells from a non-haematopoietic tissue sample comprising the steps of:
(i) isolating a population of γδ T cells from a non-haematopoietic tissue sample according to the method defined herein; and
(ii) culturing said population of γδ T cells in the presence of:
   (a) IL-2 or IL-9;
   (b) IL15; and
   (c) IL-21; and
   (d) IL-4
for at least 5 days in amounts effective to produce an expanded population of γδ T cells.

According to one aspect of the invention, there is provided an expanded population of isolated lymphocytes (e.g. skin-derived αβ T cells and/or NK cells) obtained by any of the methods defined herein.

According to a further aspect of the invention, there is provided an expanded population of isolated lymphocytes cells obtainable by any of the methods defined herein.

According to a yet further aspect of the invention, there is provided an expanded population of isolated γδ T cells obtained by any of the methods defined herein.

According to a yet further aspect of the invention, there is provided an expanded population of isolated γδ T cells obtainable by any of the methods defined herein.

Applications for Cells

The lymphocytes and/or γδ T cells obtained by the method of the invention may be used as a medicament, for example for adoptive T cell therapy. This involves the transfer of lymphocytes and/or γδ T cells obtained by the method of the invention into a patient. The therapy may be autologous, i.e. the γδ T cells may be transferred back into the same patient from which they were obtained, or the therapy may be allogeneic, i.e. the γδ T cells from one person may be transferred into a different patient. In instances involving allogeneic transfer, the γδ T cells may be substantially free of αβ T cells. For example, αβ T cells may be depleted from the γδ T cell population, e.g., after expansion, using any suitable means known in the art (e.g., by negative selection, e.g., using magnetic beads). A method of treatment may include; providing a sample of non-haematopoietic tissue obtained from a donor individual; isolating γδ T cells from the non-haematopoietic tissue sample as described herein; culturing the isolated γδ T cells to produce an expanded population; and administering the expanded population of γδ T cells to a recipient individual.

The patient or subject to be treated is preferably a human cancer patient (e.g., a human cancer patient being treated for a solid tumor) or a virus-infected patient (e.g., a CMV-infected or HIV infected patient). In some instances, the patient has and/or is being treated for a solid tumor.

Because they are normally resident in non-haematopoietic tissues, tissue-resident Vδ1 T and DN γδ T cells are also more likely to home to and be retained within tumor masses than their systemic blood-resident counterparts and adoptive transfer of these cells is likely to be more effective at targeting solid tumors and potentially other non-haematopoietic tissue-associated immunopathologies.

As γδ T cells are non-MHC restricted, they do not recognize a host into which they are transferred as foreign, which means that they are less likely to cause graft-versus-host disease. This means that they can be used “off the shelf” and transferred into any recipient, e.g., for allogeneic adoptive T cell therapy.

Non-haematopoietic tissue-resident γδ T cells obtained by methods of the invention express NKG2D and respond to a NKG2D ligand (e.g. MICA), which is strongly associated with malignancy. They also express a cytotoxic profile in the absence of any activation and are therefore likely to be effective at killing tumor cells. For example, the non-haematopoietic tissue-resident γδ T cells obtained as described herein may express one or more, preferably all of IFN-γ, TNF-α, GM-CSF, CCL4, IL-13, Granulysin, Granzyme A and B, and Perforin in the absence of any activation. IL-17A may not be expressed.

There is compelling evidence for the practicality and suitability for the clinical application of the non-haematopoietic tissue-resident γδ T cells obtained by the method of the invention as an “off-the-shelf” immunotherapeutic reagent. These cells possess innate-like killing, have no MHC restriction and display improved homing to and/or retention within tumors than do other T cells.

In some embodiments, a method of treatment of an individual with a tumor in a non-haematopoietic tissue may include; providing a sample of said non-haematopoietic tissue obtained from a donor individual, culturing the γδ T cells from the sample as described above to produce an expanded population, and; administering the expanded population of γδ T cells to the individual with the tumor.

Pharmaceutical compositions may include expanded non-haematopoietic tissue-resident γδ T cells as described herein in combination with one or more pharmaceutically or physiologically acceptable carrier, diluents, or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Cryopreservation solutions which may be used in the pharmaceutical compositions of the invention include, for example, DMSO. Compositions can be formulated, e.g., for intravenous administration.

In one embodiment, the pharmaceutical composition is substantially free of, e.g., there are no detectable levels of a contaminant, e.g., of endotoxin or mycoplasma.

In some instances, a therapeutically effective amount of expanded γδ T cells obtained by the any of the methods described above can be administered in a therapeutically effective amount to a subject (e.g., for treatment of cancer, e.g. for treatment of a solid tumor). In some cases, the therapeutically effective amount of expanded γδ T cells (e.g., skin-derived γδ T cells and/or non-Vδ2 T cells, e.g., Vδ1 T cells and/or DN T cells) is less than 10×10¹² cells per dose (e.g., less than 9×10¹² cells per dose, less than 8×10¹² cells per dose, less than 7×10¹² cells per dose, less than 6×10¹² cells per dose, less than 5×10¹² cells per dose, less than 4×10¹² cells per dose, less than 3×10¹² cells per dose, less than 2×10¹² cells per dose, less than 1×10¹² cells per dose, less than 9×10¹¹ cells per dose, less than 8×10¹¹ cells per dose, less than 7×10¹¹ cells per dose, less than 6×10¹¹ cells per dose, less than 5×10¹¹ cells per dose, less than 4×10¹¹ cells per dose, less than 3×10¹¹ cells per dose, less than 2×10¹¹ cells per dose, less than 1×10¹¹ cells per dose, less than 9×10¹⁰ cells per dose, less than 7.5×10¹⁰ cells per dose, less than 5×10¹⁰ cells per dose, less than 2.5×10¹⁰ cells per dose, less than 1×10¹⁰ cells per dose, less than 7.5×10⁹ cells per dose, less than 5×10⁹ cells per dose, less than 2.5×10⁹ cells per dose, less than 1×10⁹ cells per dose, less than 7.5×10⁸ cells per dose, less than 5×10⁸ cells per dose, less than 2.5×10⁸ cells per dose, less than 1×10⁸ cells per dose, less than 7.5×10⁷ cells per dose, less than 5×10⁷ cells per dose, less than 2.5×10⁷ cells per dose, less than 1×10⁷ cells per dose, less than 7.5×10⁶ cells per dose, less than 5×10⁶ cells per dose, less than 2.5×10⁶ cells per dose, less than 1×10⁶ cells per dose, less than 7.5×10⁵ cells per dose, less than 5×10⁵ cells per dose, less than 2.5×10⁵ cells per dose, or less than 1×10⁵ cells per dose). In some embodiments, the therapeutically effective amount of expanded γδ T cells (e.g., skin-derived γδ T cells and/or non-Vδ2 T cells, e.g., Vδ1 T cells and/or DN T cells) is less than 10×10¹² cells over the course of treatment (e.g., less than 9×10¹² cells, less than 8×10¹² cells, less than 7×10¹² cells, less than 6×10¹² cells, less than 5×10¹² cells, less than 4×10¹² cells, less than 3×10¹² cells, less than 2×10¹² cells, less than 1×10¹² cells, less than 9×10¹¹ cells, less than 8×10¹¹ cells, less than 7×10¹¹ cells, less than 6×10¹¹ cells, less than 5×10¹¹ cells, less than 4×10¹¹ cells, less than 3×10¹¹ cells, less than 2×10¹¹ cells, less than 1×10¹¹ cells, less than 9×10¹⁰ cells, less than 7.5×10¹⁰ cells, less than 5×10¹⁰ cells, less than 2.5×10¹⁰ cells, less than 1×10¹⁰ cells, less than 7.5×10⁹ cells, less than 5×10⁹ cells, less than 2.5×10⁹ cells, less than 1×10⁹ cells, less than 7.5×10⁸ cells, less than 5×10⁸ cells, less than 2.5×10⁸ cells, less than 1×10⁸ cells, less than 7.5×10⁷ cells, less than 5×10⁷ cells, less than 2.5×10⁷ cells, less than 1×10⁷ cells, less than 7.5×10⁶ cells, less than 5×10⁶ cells, less than 2.5×10⁶ cells, less than 1×10⁶ cells, less than 7.5×10⁵ cells, less than 5×10⁵ cells, less than 2.5×10⁵ cells, or less than 1×10⁵ cells over the course of treatment).

In some embodiments, a dose of expanded non-haematopoietic tissue-resident γδ T cells as described herein comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of expanded non-haematopoietic tissue-resident γδ T cells (e.g., skin-derived γδ T cells and/or non-Vδ2 T cells, e.g., Vδ1 T cells and/or DN T cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of expanded non-haematopoietic tissue-resident γδ T cells (e.g., skin-derived γδ T cells and/or non-Vβ2 T cells, e.g., Vδ1 T cells and/or DN T cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of expanded non-haematopoietic tissue-resident γδ T cells (e.g., skin-derived γδ T cells and/or non-Vβ2 T cells, e.g., Vδ1 T cells and/or DN T cells) comprises about 1.1×10⁶-1.8×10⁷ cells/kg. In some embodiments, a dose of expanded non-haematopoietic tissue-resident γδ T cells (e.g., skin-derived γδ T cells and/or non-Vδ2 T cells, e.g., Vδ1 T cells and/or DN T cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of expanded non-haematopoietic tissue-resident γδ T cells (e.g., skin-derived γδ T cells and/or non-Vβ2 T cells, e.g., Vδ1 T cells and/or DN T cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of expanded non-haematopoietic tissue-resident γδ T cells (e.g., skin-derived γδ T cells and/or non-Vβ2 T cells, e.g., Vδ1 T cells and/or DN T cells) comprises up to about 1×10⁷, 2×10⁷, 5×10⁷, 1×10⁸, 2×10⁸, 5×10⁸, 1×10⁹, 2×10⁹, or 5×10⁹ cells.

In one embodiment, the subject is administered 10⁴ to 10⁶ expanded non-haematopoietic tissue-resident γδ T cells (e.g., skin-derived γδ T cells and/or non-Vβ2 T cells, e.g., Vδ1 T cells and/or DN T cells) per kg body weight of the subject. In one embodiment, the subject receives an initial administration of a population of non-haematopoietic tissue-resident γδ T cells (e.g., an initial administration of 10⁴ to 10⁶ γδ T cells per kg body weight of the subject, e.g., 10⁴ to 10⁵ γδ T cells per kg body weight of the subject), and one or more (e.g., 2, 3, 4, or 5) subsequent administrations of expanded non-haematopoietic tissue-resident γδ T cells (e.g., one or more subsequent administration of 10⁴ to 10⁶ expanded non-haematopoietic tissue-resident γδ T cells per kg body weight of the subject, e.g., 10⁴ to 10⁵ expanded non-haematopoietic tissue-resident γδ T cells per kg body weight of the subject). In one embodiment, the one or more subsequent administrations are administered less than 15 days, e.g., 14, 13, 12, 11, 10, 9, 8, 7, 6, 5, 4, 3, or 2 days after the previous administration, e.g., less than 4, 3, or 2 days after the previous administration. In one embodiment, the subject receives a total of about 10⁶ γδ T cells per kg body weight of the subject over the course of at least three administrations of a population of γδ T cells, e.g., the subject receives an initial dose of 1×10⁵ γδ T cells, a second administration of 3×10⁵ γδ T cells, and a third administration of 6×10⁵ γδ T cells, and, e.g., each administration is administered less than 4, 3, or 2 days after the previous administration.

The non-haematopoietic tissue-resident γδ T cells obtained by the method of the invention may also be gene engineered for enhanced therapeutic properties, such as for CAR-T therapy. This involves the generation of engineered T cell receptors (TCRs) to re-program the T cell with a new specificity, e.g. the specificity of a monoclonal antibody. The engineered TCR may make the T cells specific for malignant cells and therefore useful for cancer immunotherapy. For example, the T cells may recognize cancer cells expressing a tumor antigen, such as a tumor associated antigen that is not expressed by normal somatic cells from the subject tissue. Thus, the CAR-modified T cells may be used for adoptive T cell therapy of, for example, cancer patients.

The use of blood-resident γδ T cells for CAR has been described. However, non-haematopoietic tissue-resident γδ T cells obtained by the method of the invention are likely to be particularly good vehicles for CAR-T approaches, as they can be transduced with chimeric antigen-specific TCRs while retaining their innate-like capabilities of recognizing transformed cells, and are likely to have better tumor penetration and retention capabilities than either blood-resident γδ T cells or conventional, systemic αβ T cells. Furthermore, their lack of MHC dependent antigen presentation reduces the potential for graft-versus-host disease and permits them to target tumors expressing low levels of MHC. Likewise, their non-reliance upon conventional co-stimulation, for example via engagement of CD28 enhances the targeting of tumors expressing low levels of ligands for co-stimulatory receptors.

In some embodiments, one or more additional therapeutic agents can be administered to the subject. The additional therapeutic agent may be selected from the group consisting of an immunotherapeutic agent, a cytotoxic agent, a growth inhibitory agent, a radiation therapy agent, an anti-angiogenic agent, or a combination of two or more agents thereof. The additional therapeutic agent may be administered concurrently with, prior to, or after administration of the expanded γδ T cells. The additional therapeutic agent may be an immunotherapeutic agent, which may act on a target within the subject's body (e.g., the subject's own immune system) and/or on the transferred γδ T cells.

The administration of the compositions may be carried out in any convenient manner. The compositions described herein may be administered to a patient transarterially, subcutaneously, intradermally, intratumorally, intranodally, intramedullary, intramuscularly, by intravenous injection, or intraperitoneally, e.g., by intradermal or subcutaneous injection. The compositions of non-haematopoietic tissue-resident γδ T cells may be injected directly into a tumor, lymph node, or site of infection.

It will be understood that all embodiments described herein may be applied to all aspects of the invention.

As used herein, the term “about” when used herein includes up to and including 10% greater and up to and including 10% lower than the value specified, suitably up to and including 5% greater and up to and including 5% lower than the value specified, especially the value specified. The term “between”, includes the values of the specified boundaries.

Certain aspects and embodiments of the invention will now be illustrated by way of example and with reference to the figures described above.

### EXAMPLES

### Example 1. Methods and Materials

Unless otherwise stated, the following methods were utilized to generate the results of the subsequent examples.

Materials

Sources of materials used in the experimental conditions are summarised in Table 1.

TABLE 1
Materials used in the study

| Reagent | Supplier |
|---|---|
| AIM-V | Fisher |
| CTS Immune Serum Replacement | Life Technologies |
| Human Serum Albumin | Sigma-Aldrich |
| Human Plasma | Merck Chemicals Ltd. |
| Human IL-2, premium grade | Miltenyi |
| Human IL-15, premium grade | Miltenyi |
| Human IL-4, premium grade | Miltenyi |
| Human IL-21 | Peprotech |
| Human IFN-γ | Peprotech |
| Human IL-1β | Peprotech |

Design of Experiment Modelling

Design of experiments (DoE) was generated using JMP software 15.0. DoE custom design was applied to generate an experiment to analyse the effects of protein source, IL-1β, IFN-γ, IL-21 and IL-4 on defined outcomes, such as γδ T cell enrichment. Protein source was treated as a categorical variable and IL-1β, IFN-γ, IL-21 and IL-4 were treated as continuous variables. In addition, JMP scripts were used to disallow experimental conditions to contain IL-4 alone.

A factorial to degree 2 design was applied to enable linear and 2nd degree interactions to be determined (e.g. IL-21* IL-1β). A design of 21 conditions was taken forward for experimental testing.

Upon collection of experimental data, outlier removal was applied by calculation of studentised residuals based on % γδ T cells as the response readout. Any rows yielding a studentised residual of >4 or <4 for % γδ T cells were excluded from the analysis. A total of 7 rows out of 84 were excluded.

After removal of outliers, standard least squares fitting was applied to a given response based the value for that given response from each donor. To account for donor variation, donor ID was added as a stand-alone variable into the least squares fit model. Protein source and cytokine interaction p-values relating a selected outcome, such as % γδ T cells or % TIGIT+γδ T cells, were eliminated in a step-by-step method until only interactions with p<0.05 remained.

Isolation Setup and Harvest

Before the sample arrives, relevant media are prepared and 20 mm×1.5 mm carbon matrices (‘grids’) (Cytomatrix Pty Ltd, Australia or Ultramet, USA) are autoclaved. Grids are then rinsed in phosphate-buffered saline (PBS) and submerged in PBS until use.

Preparation of Organotypic Skin Culture

Skin samples are prepared by removing subcutaneous fat using forceps, scissors and scalpel. Taking one piece of tissue at a time, a 3 mm biopsy punch is used to make multiple punches. Three tissue punches are placed on each grid.

One grid is placed per well of a G-REX6 (Wilson Wolf). Each well is filled with 5 ml of relevant base media and 5 ml of 2× relevant condition media (resulting in 10 ml of 1× relevant condition media). 100 μL of Amphotericin B (Life Technologies) is added to each well (resulting in 1% Amphotericin B). Plates are incubated at 37° C. and 5% CO₂ for 21 days. Culture is fed on day 7 and day 14 by gently adding 10 ml of 2× relevant media to each well.

Isolation Harvest

Matrices are removed. Cells are resuspended in each well using a pipette and transferred to a centrifuge tube. Wells are washed with PBS and the wash is also transferred to the centrifuge tube. Isolated cells may then be subsequently analysed, e.g. for cell count or in flow cytometric analysis.

Freezing of Isolated Cells

Once the isolated cells have been analysed, cell suspensions are spun down in a centrifuge. The supernatant is discarded and cell pellet resuspended in Cryostor10 cell freezing solution (Sigma Aldrich) to a final concentration of 100×10⁶ cells per ml. Cell suspension is then transferred into freezing vials and placed into a cool cell or Mr Frosty freezing unit (Thermo Scientific) and placed in −80° C. freezer overnight. The next day, cells are transferred to liquid nitrogen storage (vapour phase).

Flow Cytometry

Flow cytometry was performed using the following antibody-fluorochrome conjugates:

a) Batch Release panel: CD45-FITC, CD25-PE, PANαβ-PerCP Vio700, NKG2D-PEVio770, Vδ1-Vioblue, PANγδ-APC.

b) Functional panel: CD45RA-FITC, TIGIT-PE, PANαβ-PerCP Vio700, NKG2A-PEVio770, Vδ1-Vioblue, PD1-BVδ10, PANγδ-APC.

Commercial antibodies were purchased from Biolegend or Miltenyi. Viability dye (eFluor780) was from ThermoFischer Scientific. Flow cytometry data analysis was performed on FLOWJO (Version 10.6.2).

Determining Total Cell Number

Total cell numbers were generated using an NC-250 Nucleocounter (Chemometec, Copenhagen Denmark) and manufacturer's instructions.

### Example 2. Isolation Optimisation Experiment

Optimisation of the protocol for isolating γδ T cells from non-haematopoietic tissue samples was investigated to improve γδ T cell yield and potentially improve Vδ1 T cell quality at isolation. Improving the starting material will help to improve yield and quality in subsequent expansion steps.

The Design of Experiment (DoE) investigated the impact of different protein sources and cytokines on the skin isolation culture. In order to make a feasible design, media type, IL-15 concentration, and IL-2 concentration were kept constant. Different combinations of IL-21, IFN-γ, IL-1β were investigated. Addition of IL-4 was also investigated, although only if IL-21, IFN-γ or IL-1β were present.

All cells were isolated in AIM-V media supplements with either 5% serum replacement (SR), 2.5% allogeneic plasma or 10% allogeneic AB male serum. All cultures were setup with IL-2 and IL-15, and supplemented with varying combinations of IL-4, IL-21, IFN-γ, IL-1β. The tested culture conditions are summarised in Table 2.

TABLE 2
Summary of experimental conditions tested

| | | | IL-15 | IL-2 | IL-21 | IFN-γ | IL-1b | IL-4 |
|---|---|---|---|---|---|---|---|---|
| Run | Protein | | (U/mL) | (U/mL) | (U/mL) | (U/mL) | (U/mL) | (U/mL) |
| 1 | Plasma | (2.5%) | 600 | 138 | 1.05 | 0 | 0 | 95 |
| 2 | SR | (5%) | 600 | 138 | 1.05 | 420 | 4500 | 0 |
| 3 | SR | (5%) | 600 | 138 | 0 | 0 | 4500 | 0 |
| 4 | AB | (10%) | 600 | 138 | 0 | 420 | 0 | 0 |
| 5 | AB | (10%) | 600 | 138 | 1.05 | 0 | 0 | 95 |
| 6 | AB | (10%) | 600 | 138 | 0 | 0 | 4500 | 95 |
| 7 | Plasma | (2.5%) | 600 | 138 | 1.05 | 420 | 4500 | 95 |
| 8 | AB | (10%) | 600 | 138 | 1.05 | 420 | 4500 | 95 |
| 9 | Plasma | (2.5%) | 600 | 138 | 0 | 420 | 4500 | 0 |
| 10 | SR | (5%) | 600 | 138 | 1.05 | 0 | 0 | 0 |
| 11 | SR | (5%) | 600 | 138 | 0 | 420 | 0 | 0 |
| 12 | AB | (10%) | 600 | 138 | 1.05 | 0 | 4500 | 0 |
| 13 | SR | (5%) | 600 | 138 | 1.05 | 420 | 0 | 95 |
| 14 | Plasma | (2.5%) | 600 | 138 | 0 | 0 | 4500 | 95 |
| 15 | Plasma | (2.5%) | 600 | 138 | 1.05 | 420 | 0 | 0 |
| 16 | SR | (5%) | 600 | 138 | 0 | 420 | 4500 | 95 |
| 17 | Plasma | (2.5%) | 600 | 138 | 0 | 420 | 0 | 95 |
| 18 | Plasma | (2.5%) | 600 | 138 | 0 | 0 | 0 | 0 |
| 19 | Plasma | (2.5%) | 600 | 138 | 1.05 | 0 | 4500 | 0 |
| 20 | SR | (5%) | 600 | 138 | 1.05 | 0 | 4500 | 95 |
| 21 | SR | (5%) | 600 | 138 | 1.05 | 0 | 0 | 95 |
| STD ISO | SR | (5%) | 600 | 138 | 0 | 0 | 0 | 0 |

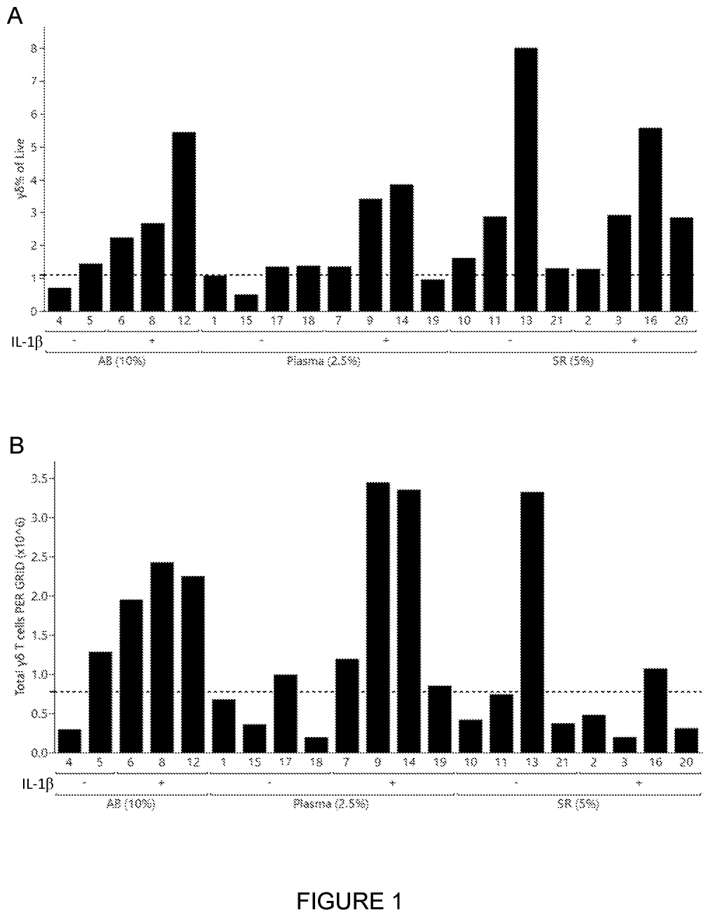

Results from the culture conditions are presented in FIGS. 1-10.

Cytokines

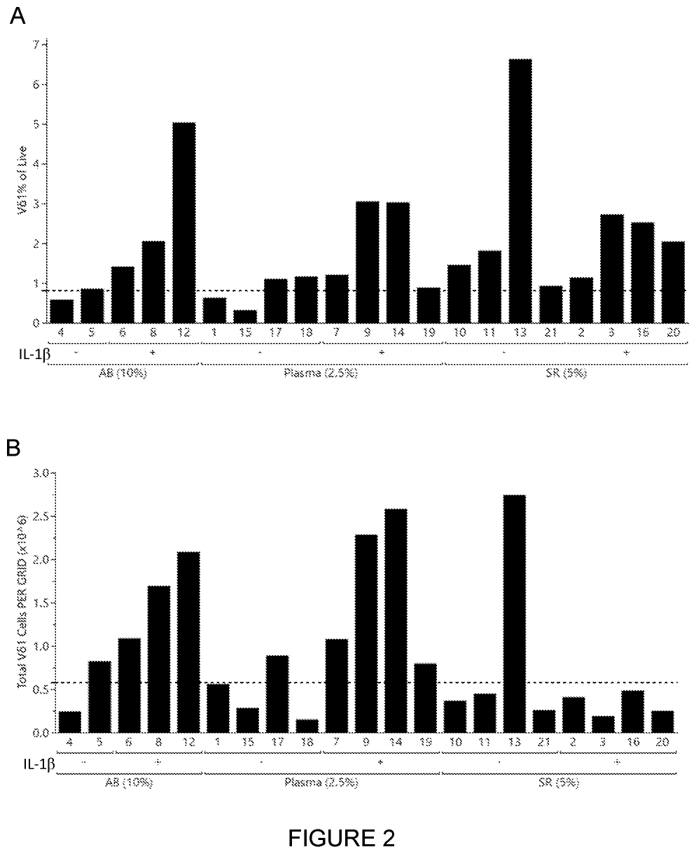

The use of IL-1β, particularly in the context of plasma and AB serum isolations, had the effect of increasing the overall yield of γδ T cells and the overall yield of Vδ1 T cells isolated. Results of % and total γδ T cells for all conditions are shown in FIG. 1 and % and total Vδ1 T cells are shown in FIG. 2.

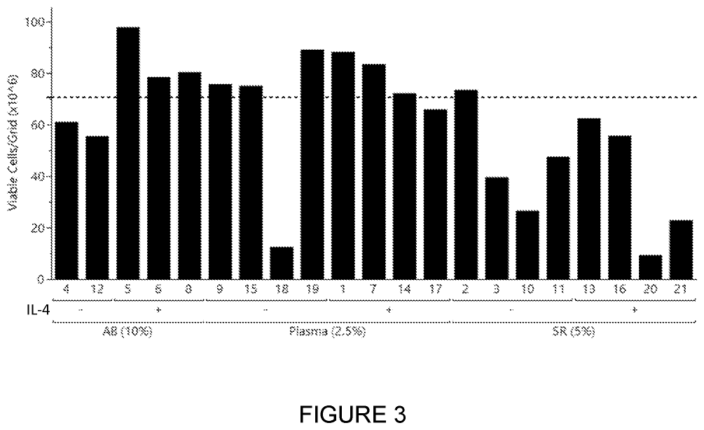

The addition of IL-4 to AB serum had the effect of increasing the overall number of cells isolated per grid (FIG. 3). A more moderate increase was seen in plasma isolated cultures.

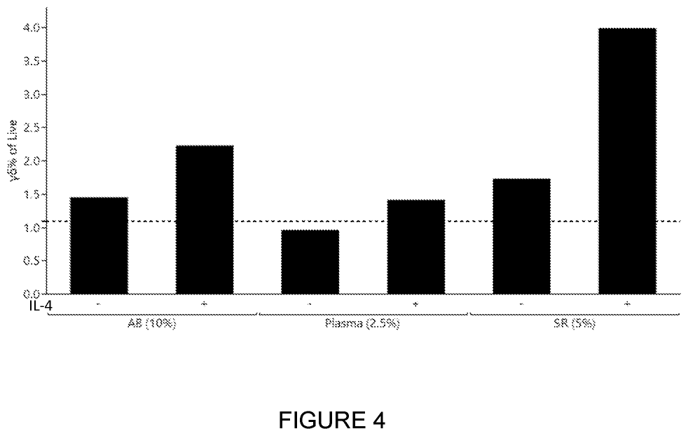

The addition of IL-4 to plasma, SR or AB serum samples had the effect of increasing the % γδ enrichment in cultures (FIG. 4).

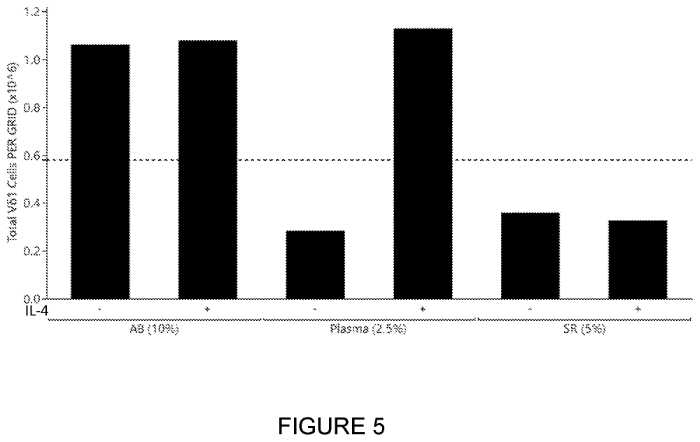

The addition of IL-4 to plasma cultures had the effect of increasing the overall number of Vδ1 cells isolated per grid (FIG. 5). The same effect was not observed in SR or human AB serum cultures; however the use of AB serum increased the overall number of Vδ1 cells isolated per grid compared to the control.

The addition of IL-21 to AB serum cultures had the effect of increasing the overall number of γδ T cells isolated. However, the benefit of IL-21 was not seen in cultures isolated with plasma or SR. DoE modelling for % γδ T cell for each donor in plasma with IL-1β and IFN-γ indicates that IL-1β is most beneficial when IFN-γ and IL-21 are absent.

Phenotype Analysis

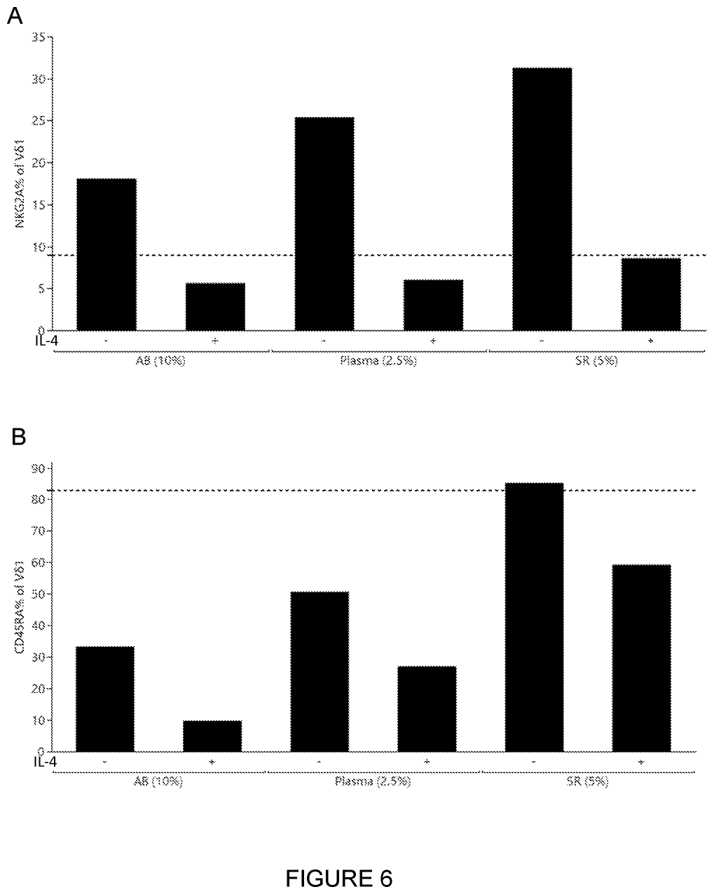

The addition of IL-4 to both AB serum and plasma cultures had the effect of decreasing the expression of both NKG2A and CD45RA on Vδ1 T cells (FIG. 6).

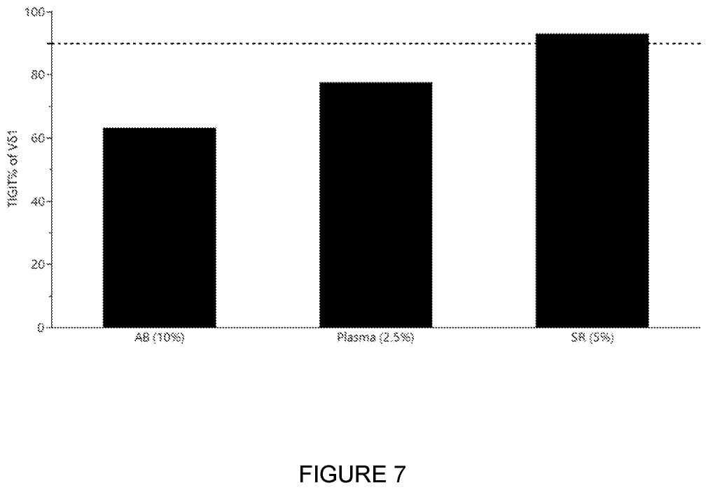

The use of SR resulted in increased expression of the checkpoint inhibitor marker TIGIT on Vδ1 T cells relative to both plasma and AB serum isolation cultures (FIG. 7).

Analysis also showed that use of SR resulted in increased expression of the terminal differentiation marker CD45RA and the inhibitory receptor NKp44 on Vδ1 T cells relative to both plasma and AB serum (data not shown).

Protein Source

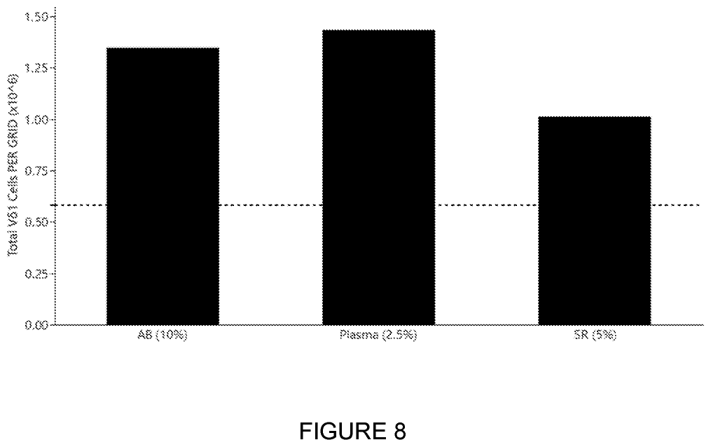

The use of both plasma and AB serum in culture has the effect of increasing the overall number of Vδ1 T cells per grid relative to SR culture isolations (FIG. 8). However, DoE modelling showed that the addition of IFN-γ to SR isolated samples increased the overall number of γδ T cells isolated.

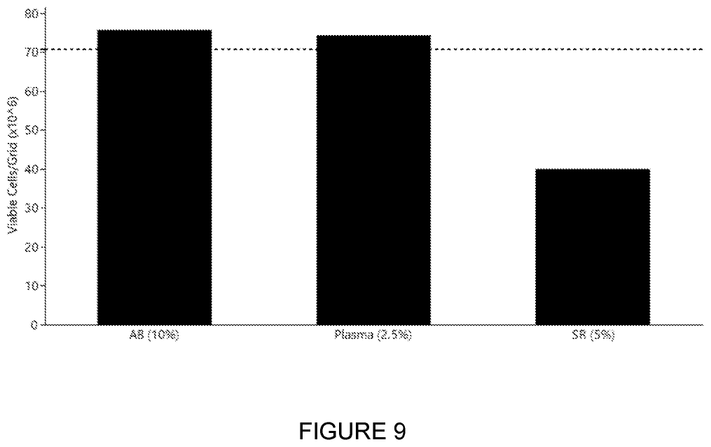

The use of SR also appears to result in lower overall viability of isolated cells compared to plasma or AB serum as a protein source (FIG. 9).

Other Lymphocytes

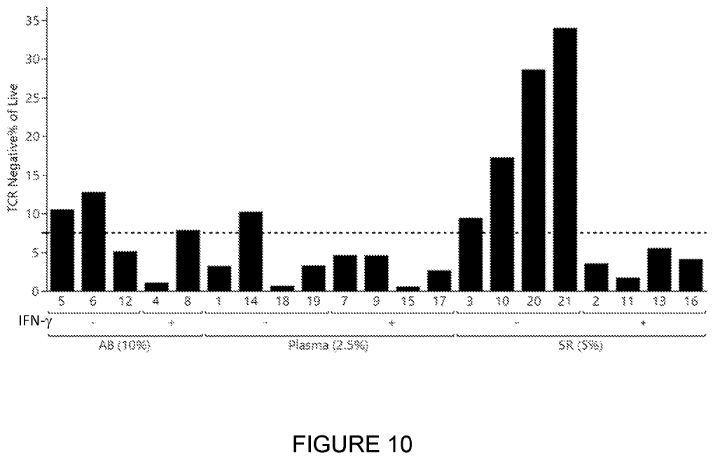

The conditions were also investigated for the generation of TCR-negative cells (i.e. cells which are negative for αβ-TCR and γδ-TCR). The use of SR as the protein source results in increased numbers of TCR-negative cells compared to plasma or AB serum (FIG. 10). Conditions where IFN-γ is absent also increased TCR-negative cell number.

Conclusion

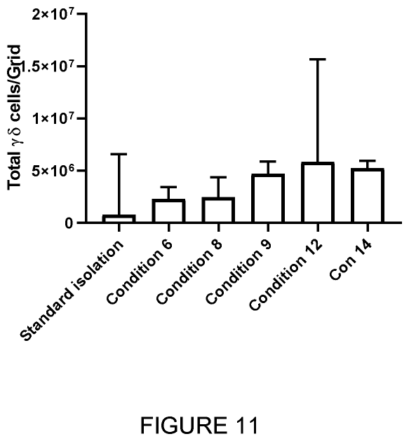

The data suggests the overall number of γδ cells isolated can be vastly improved which provides the potential to increase initial number of expansion setups. All of the conditions with the best % γδ T cells and total γδ T cells per grid contained IL-1β (conditions 6, 8, 9, 12 and 14). Results from the top conditions compared to the standard 2 cytokine isolation method are shown in FIG. 11.

Therefore, through DoE approaches titrating a combination of human cytokines and either human AB serum, human pool plasma or serum replacement, a skin lymphocyte isolation cocktail was identified termed Generation 2, using human pool plasma (previous isolation cultures used serum replacement) and the cytokines IL-2, IL-4, IL-15 and IL-1β (only IL-2 and IL-15 were used previously). This new growth cocktail, compared to the original methodology (now also known as Generation 1), improved the number of total viable lymphocytes isolated from 50-100×10⁶ cells (previously) to 75-200×10⁶ lymphocytes per isolation cell matrix (grid). Generation 2 furthermore showed an up to 3-fold higher presence of γδ T cells at the end of isolation and in general presented with a favourable phenotype (e.g. lower CD45RA and NKG2A expression).

### Example 3. Investigating Frozen Isolations

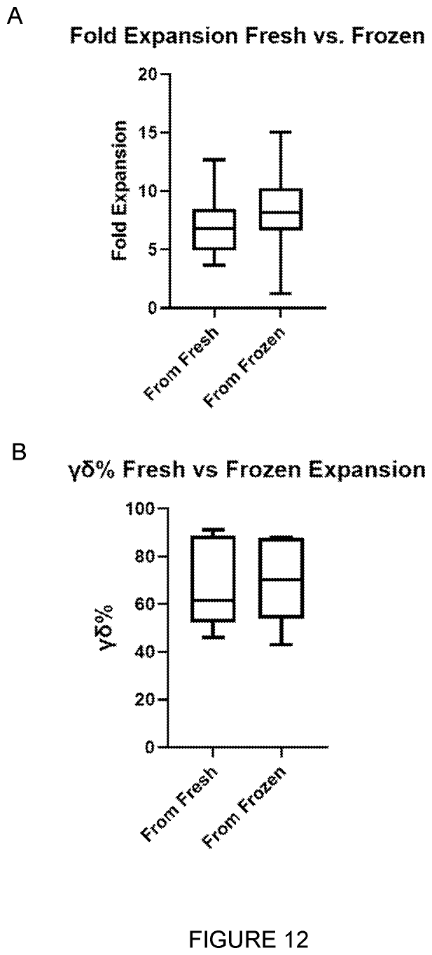

Previous conditions have resulted in large numbers of γδ T cells, but poor viability after harvesting and thawing. A new isolation and expansion process was investigated where the isolated cells were frozen prior to expansion. After isolation, cells were frozen according to the method detailed in Example 1. The effects on fold expansion and % γδ T cell in subsequent expansion steps are shown in FIG. 12. It was found that isolated cells that were frozen prior to expansion, enrich and expand at least as well as fresh equivalents.

### Example 4. The Inclusion of IL-21 in the Isolation Formulation May Increase γδ T Cell Yield

The use of IL-21 was investigated to examine if this cytokine could increase the overall γδ T cell yield following isolation and subsequent expansion. Improving the overall γδ T cell content would enable possible larger-scale gene engineering of these cells in subsequent expansions as well potentially increasing the overall effector cell yield.

Isolation cultures were set up in GREX100M units with AIMV media supplemented with 2.5% allogenic plasma (see Example 1 for details). Cultures were then supplemented with cytokines as detailed in Table 3.

TABLE 3
Cytokine conditions used for isolation cultures

| | “No IL-21” variation | “IL-21” variation (18.8 ng/mL; |
|---|---|---|
| | (Generation 2) | Generation 3) |
| IL-2 (U/ml) | 138 | 138 |
| IL-4 (U/ml) | 95 | 32 |
| IL-15 (U/ml) | 600 | 600 |
| IL-1β (U/ml) | 4500 | 4500 |
| IL-21 (ng/ml) | 0 | 18.8 |

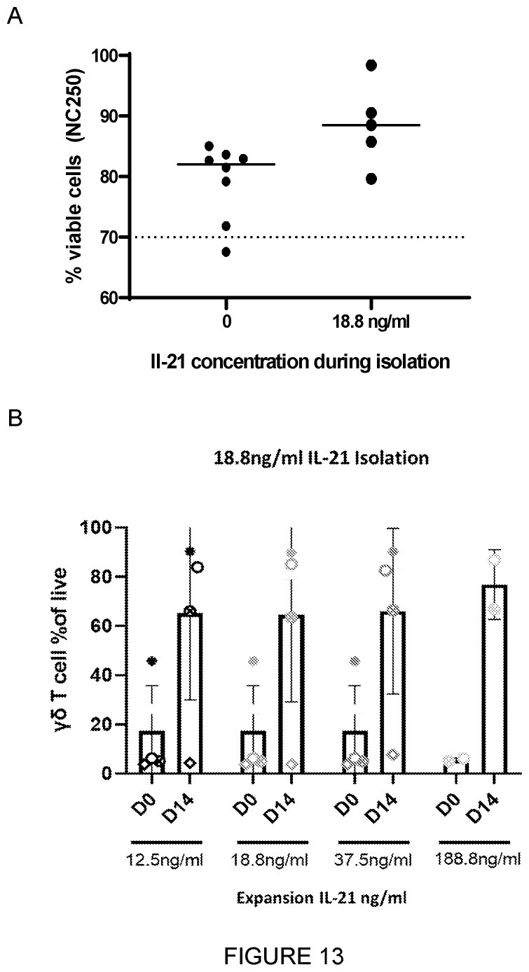

Isolation cultures with 18.8 ng/ml IL-21 were harvested at day 21 and cryopreserved (frozen) for subsequent expansion. Cells were then thawed and cell viability was measured via NC250 automatic cell counting and the results are shown in FIG. 13A. The overall viability of cells at the point of thawing was above the minimum acceptable viability both with and without IL-21, with a mean % viability >80% in “No IL-21” cultures and nearly 90% in “IL-21” cultures. The isolated cells were then expanded in the presence of TexMACS™ media (Miltenyi Biotec) supplemented with 5% allogeneic plasma and IL-15 (80 ng/mL) with different levels of IL-21 supplementation (12.53, 18.8, 37.5 and 188 ng/mL). After expanding for 14 days, the cells were harvested and γδ T cell enrichment measured via flow cytometry as shown in FIG. 13B. After expansion, the cells that had been isolated in the presence of IL-21 demonstrated γδ T cell enrichment over the course of the 14 day culture, independent of the amounts of IL-21 used for expansion showing comparable performance from 12.5 to 188.8 ng/mL. Therefore, the presence of IL-21 in the isolation culture yields viable cells and maintains the ability for γδ T cells to be subsequently expanded. The functionality of these cells post-expansion in cytotoxicity assays was also confirmed.

### Example 5. Comparison of Isolation Cultures with No IL-21 Versus 18.8 ng/ml IL-21

Donor-matched skin samples were set up in isolation cultures with either the “No IL-21” formulation or the “IL-21” (18.8 ng/ml) cytokine formulation as detailed in Table 3. All isolation cultures were set up with AIMV media supplemented with 2.5% allogeneic plasma (see Example 1 for details).

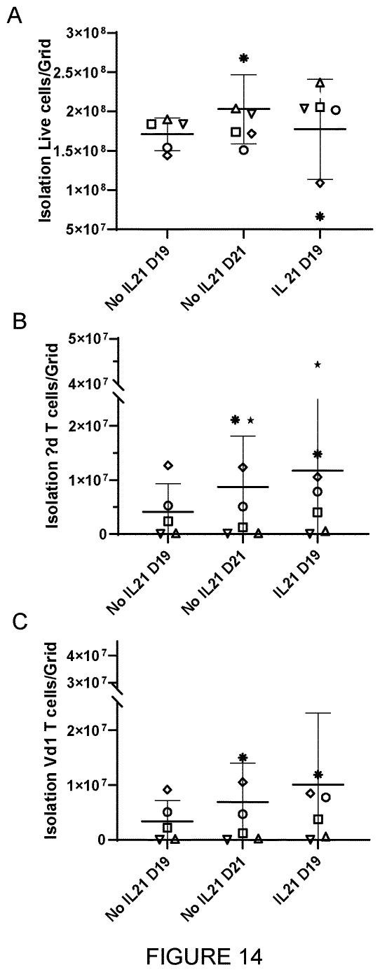

“No IL-21” isolation cultures were harvested at either day 19 or day 21, while “IL-21” isolation cultures were harvested at day 19 only. At the point of harvest, the total number of viable cells per culture grid, total number of isolated γδ T cells per isolation culture grid and total number of Vδ1 T cells per isolation culture grid were recorded and the results are shown in FIG. 14.

1. A method for the isolation of lymphocytes from a non-haematopoietic tissue sample comprising the steps of:
(i) culturing the non-haematopoietic tissue sample in the presence of Interleukin-1β (IL-1β); and
(ii) collecting a population of lymphocytes cultured from the non-haematopoietic tissue sample.

2. A method for the isolation of γδ T cells from a non-haematopoietic tissue sample comprising the steps of:
(i) culturing the non-haematopoietic tissue sample in the presence of IL-1β; and
(ii) collecting a population of γδ T cells cultured from the non-haematopoietic tissue sample.

3. The method according to any preceding claim, wherein step (i) further comprises culturing the non-haematopoietic tissue sample in the presence of Interleukin-2 (IL-2) and Interleukin-15 (IL-15).

4. The method according to any preceding claim, wherein step (i) further comprises culturing the non-haematopoietic tissue sample in the presence of Interleukin-4 (IL-4) and/or Interferon-γ (IFN-γ).

5. The method according to any preceding claim, wherein the culturing is performed in the absence of Interleukin-21 (IL-21).

6. The method according to any one of claims 1 to 4, wherein the culturing is performed in the presence Interleukin-21 (IL-21) at a concentration between 15 ng/mL and 25 ng/mL, such as 18 to 20 ng/ml.

7. The method according to any preceding claim, wherein the non-haematopoietic tissue sample is cultured in media containing serum or plasma.

8. The method according to claim 7, wherein the non-haematopoietic tissue is cultured in media containing 2.5% human plasma.

9. The method according to claim 7, wherein the non-haematopoietic tissue is cultured in media containing 10% human AB serum.

10. The method according to any preceding claim, wherein the lymphocytes or γδ T cells are collected after at least 7 days of culturing.

11. The method according to any preceding claim, wherein the lymphocytes or γδ T cells are collected after at least 14 days of culturing.

12. The method according to any preceding claim, wherein the lymphocytes or γδ T cells are collected prior to 35 days of culturing.

13. The method according to any preceding claim, wherein the lymphocytes or γδ T cells are collected after 19 days of culturing.

14. The method according to any preceding claim, wherein the lymphocytes or γδ T cells are collected after 21 days of culturing.

15. The method according to any preceding claim, wherein the non-haematopoietic tissue sample is an intact biopsy.

16. The method according to any preceding claim, wherein the non-haematopoietic tissue sample is skin.

17. The method according to claim 16, wherein the skin sample comprises the epidermal and dermal layer.

18. The method according to any preceding claim, wherein the non-haematopoietic tissue sample is gut or gastrointestinal tract.

19. The method according to any preceding claim, wherein the non-haematopoietic tissue sample has been obtained from a human.

20. The method according to any preceding claim, wherein the method is performed in a vessel comprising a gas permeable material.

21. The method according to any preceding claim, wherein the isolated population of lymphocytes or γδ T cells comprise a population of Vδ1 T cells.

22. The method according to claim 21, wherein less than 80% of the isolated population of Vδ1 T cells expresses CD45RA, such as less than 30% of the isolated population of Vδ1 T cells expresses CD45RA.

23. The method according to claim 21 or claim 22, wherein less than 10% of the isolated population of Vδ1 T cells expresses NKG2A.

24. The method according to any preceding claim, further comprising expanding the isolated population of lymphocytes or γδ T cells.

25. A method for the isolation of lymphocytes from a non-haematopoietic tissue sample comprising the steps of:
(i) culturing the non-haematopoietic tissue sample in media comprising serum replacement, human plasma and/or human AB serum; and
(ii) collecting a population of lymphocytes cultured from the non-haematopoietic tissue sample.

26. The method according to claim 25, comprising culturing the non-haematopoietic tissue sample in media comprising serum replacement.

27. The method according to either claim 25 or 26, wherein the isolated population of lymphocytes comprise a population of TCR-negative cells.

28. The method according to any one of claims 25 to 27, wherein step (i) further comprises culturing the non-haematopoietic tissue sample in the presence of IL-2 and IL-15.

29. The method according to any one of claims 25 to 28, wherein step (i) further comprises culturing the non-haematopoietic tissue sample in the presence of IL-4 and/or IL-1β.

30. The method according to any one of claims 25 to 29, wherein the culturing is performed in the absence of IFN-γ.

31. The method according to any one of claims 25 to 30, wherein step (i) further comprises culturing the non-haematopoietic tissue sample in the presence of IL-21 at a concentration between 15 ng/mL and 25 ng/mL.

32. The method according to any one of claims 25 to 31, wherein the lymphocytes are collected after 19 days of culturing.

33. The method according to any one of claims 25 to 31, wherein the lymphocytes are collected after 21 days of culturing.

34. A method for the isolation and expansion of lymphocytes from a non-haematopoietic tissue sample comprising the steps of:
(i) isolating a population of lymphocytes from the non-haematopoietic tissue sample according to the method of any preceding claim; and
(ii) further culturing said population of lymphocytes for at least 5 days to produce an expanded population of lymphocytes.

35. A method for the isolation and expansion of γδ T cells from a non-haematopoietic tissue sample comprising the steps of:
(i) isolating a population of γδ T cells from the non-haematopoietic tissue sample according to the method of any preceding claim; and
(ii) further culturing said population of γδ T cells for at least 5 days to produce an expanded population of γδ T cells.

36. The method according to claim 34 or claim 35, wherein the isolated lymphocyte or γδ T cell population is frozen and then thawed prior to step (ii).

37. The method according to claim 35 or claim 36, wherein the expansion step comprises culturing the γδ T cells in the presence of:
(a) IL-2 or IL-9;
(b) IL-15; and optionally
(c) IL-21
for at least 5 days in amounts effective to produce an expanded population of γδ T cells.

38. The method according to claim 37, which further comprises culturing the γδ T cells in the presence of IL-4.

39. The method according to any one of claims 35 to 38, wherein the expansion step comprises culturing the γδ T cells in the absence of substantial stromal cell contact.

40. The method according to any one of claims 35 to 39, wherein the expansion step comprises the absence of exogenous TCR pathway agonists.

41. A method of isolation for γδ T cells from a non-haematopoietic tissue sample comprising the steps of:
(i) isolating a population of γδ T cells from the non-haematopoietic tissue sample; and
(ii) freezing the isolated population of γδ T cells.

42. The method according to claim 41, wherein γδ T cells are isolated by culturing the non-haematopoietic tissue sample in the presence of IL-2 and IL-15, optionally in combination with IL-1β, IL-4 and/or IL-21.

43. The method according to claim 42, wherein γδ T cells are isolated by culturing the non-haematopoietic tissue sample in the presence of IL-21 at a concentration between 15 ng/mL and 25 ng/mL, such as 18 to 20 ng/ml.

44. The method according to claims 41 to 43, wherein the γδ T cells are frozen after 19 days of isolation.

45. The method according to any one of claims 41 to 43, wherein the γδ T cells are frozen after 21 days of isolation.

46. The method according to any one of claims 41 to 44, wherein the isolated population of γδ T cells are frozen in a freezing solution and stored in liquid nitrogen (vapour phase).

47. The method according to any one of claims 41 to 45, which additionally comprises: (iii) thawing the frozen population of γδ T cells; and (iv) culturing the thawed population of γδ T cells for at least 5 days to produce an expanded population of γδ T cells.

48. An isolated γδ T cell population obtainable by the method of any one of claims 1 to 24.

49. An isolated γδ T cell population obtained by the method of any one of claims 1 to 24.

50. A frozen isolated γδ T cell population obtainable by the method of any one of claims 41 to 47.

51. A frozen isolated γδ T cell population obtained by the method of any one of claims 41 to 47.

52. An isolated and expanded γδ T cell population obtainable by the method of any one of claims 35 to 40.

52. An isolated and expanded γδ T cell population obtained by the method of any one of claims 35 to 40.
